SEQUENCE LISTING (100 sequences)

SEQ ID NO: 1 - DNA, 57 bp, synthetic construct
  misc_feature 1..57: note = Synthetic
  misc_feature 57: note = n is a, c, g, or t
  source 1..57: mol_type = other DNA
aagcagtggtatcaacgcagagtacttttttttttttttttttttttttttttttvn

SEQ ID NO: 2 - DNA, 41 bp, synthetic construct
  misc_feature 1..41: note = Synthetic
  misc_feature 39..41: note = 3' terminal ribonucleotide subsequence
  source 1..41: mol_type = other DNA
gtggtatcaacgcagagtacacgacgctcttccgatctggg

SEQ ID NO: 3 - DNA, 23 bp, synthetic construct
  misc_feature 1..23: note = Synthetic
  source 1..23: mol_type = other DNA
aagcagtggtatcaacgcagagt

SEQ ID NO: 4 - DNA, 55 bp, synthetic construct
  misc_feature 1..55: note = Synthetic
  source 1..55: mol_type = other DNA
tcgtcggcagcgtcagatgtgtataagagacagtacacgacgctcttccgatctg

SEQ ID NO: 5 - DNA, 54 bp, synthetic construct
  misc_feature 1..54: note = Synthetic
  source 1..54: mol_type = other DNA
gtctcgtgggctcggagatgtgtataagagacagacggcagggtcagggttctg

SEQ ID NO: 6 - DNA, 54 bp, synthetic construct
  misc_feature 1..54: note = Synthetic
  source 1..54: mol_type = other DNA
gtctcgtgggctcggagatgtgtataagagacaggcgacctcgggtgggaacac

SEQ ID NO: 7 - DNA, 960 bp, synthetic construct
  misc_feature 1..960: note = Synthetic
  source 1..960: mol_type = other DNA
ttgaaacccttcaaaggcagagacttgtccagcctaacctgcctgctgctcctagctcctgaggctcagggcccttggcttctgtccgctctgctcagggccctccagcgtggccactgctcagccatgctcctgctgctcgtcccagtgctcgaggtgatttttaccctgggaggaaccagagcccagtcggtgacccagcttggcagccacgtctctgtctctgaaggagccctggttctgctgaggtgcaactactcatcgtctgttccaccatatctcttctggtatgtgcaataccccaaccaaggactccagcttctcctgaagtacacatcagcggccaccctggttaaaggcatcaacggttttgaggctgaatttaagaagagtgaaacctccttccacctgacgaaaccctcagcccatatgagcgacgcggctgagtacttctgtgctgtgagtgatctcgaaccgaacagcagtgcttccaagataatctttggatcagggaccagactcagcatccggccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 8 - DNA, 933 bp, synthetic construct
  misc_feature 1..933: note = Synthetic
  source 1..933: mol_type = other DNA
ggctccaggctgctctgttgggtgctgctttgtctcctgggagcaggcccagtaaaggctggagtcactcaaactccaagatatctgatcaaaacgagaggacagcaagtgacactgagctgctcccctatctctgggcataggagtgtatcctggtaccaacagaccccaggacagggccttcagttcctctttgaatacttcagtgagacacagagaaacaaaggaaacttccctggtcgattctcagggcgccagttctctaactctcgctctgagatgaatgtgagcaccttggagctgggggactcggccctttatctttgcgccagcagccggacagggggcgtaaagaattcacccctccactttgggaacgggaccaggctcactgtgacagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 9 - DNA, 537 bp, synthetic construct
  misc_feature 1..537: note = Synthetic
  source 1..537: mol_type = other DNA
gacctgaaaaacgtgttcccacccaaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 10 - skipped

SEQ ID NO: 11 - protein, 16 aa, synthetic construct
  REGION 1..16: note = Synthetic
CAMSEWVTGTASKLTF

SEQ ID NO: 12 - protein, 15 aa, synthetic construct
  REGION 1..15: note = Synthetic
CATDSIHTNAGKSTF

SEQ ID NO: 13 - protein, 16 aa, synthetic construct
  REGION 1..16: note = Synthetic
CAMSASSGAGSYQLTF

SEQ ID NO: 14 - protein, 13 aa, synthetic construct
  REGION 1..13: note = Synthetic
CATDWTGGFKTIF

SEQ ID NO: 15 - protein, 12 aa, synthetic construct
  REGION 1..12: note = Synthetic
CALNTGFQKLVF

SEQ ID NO: 16 - protein, 11 aa, synthetic construct
  REGION 1..11: note = Synthetic
CAMTNDYKLSF

SEQ ID NO: 17 - protein, 19 aa, synthetic construct
  REGION 1..19: note = Synthetic
CATDLGIIPPVTNAGKSTF

SEQ ID NO: 18 - protein, 18 aa, synthetic construct
  REGION 1..18: note = Synthetic
CAPPRFRVSNFGNEKLTF

SEQ ID NO: 19 - protein, 14 aa, synthetic construct
  REGION 1..14: note = Synthetic
CAASVDNQGGKLIF

SEQ ID NO: 20 - protein, 15 aa, synthetic construct
  REGION 1..15: note = Synthetic
CAVQANNNAGNMLTF

SEQ ID NO: 21 - protein, 15 aa, synthetic construct
  REGION 1..15: note = Synthetic
CAVPGADQGAQKLVF

SEQ ID NO: 22 - protein, 12 aa, synthetic construct
  REGION 1..12: note = Synthetic
CAFTSGTYKYIF

SEQ ID NO: 23 - protein, 14 aa, synthetic construct
  REGION 1..14: note = Synthetic
CAVATNSGNTPLVF

SEQ ID NO: 24 - protein, 14 aa, synthetic construct
  REGION 1..14: note = Synthetic
CALGEQGGSEKLVF

SEQ ID NO: 25 - protein, 11 aa, synthetic construct
  REGION 1..11: note = Synthetic
CAGARNNDMRF

SEQ ID NO: 26 - protein, 14 aa, synthetic construct
  REGION 1..14: note = Synthetic
CATDATSGTYKYIF

SEQ ID NO: 27 - protein, 14 aa, synthetic construct
  REGION 1..14: note = Synthetic
CAASMTHAGNMLTF

SEQ ID NO: 28 - protein, 12 aa, synthetic construct
  REGION 1..12: note = Synthetic
CIVRDSNYQLIW

SEQ ID NO: 29 - protein, 16 aa, synthetic construct
  REGION 1..16: note = Synthetic
CAGGQSWDNYGQNFVF

SEQ ID NO: 30 - protein, 11 aa, synthetic construct
  REGION 1..11: note = Synthetic
CAVVSGYALNF

SEQ ID NO: 31 - protein, 13 aa, synthetic construct
  REGION 1..13: note = Synthetic
CAPRGGGADGLTF

SEQ ID NO: 32 - protein, 13 aa, synthetic construct
  REGION 1..13: note = Synthetic
CAAASGGSYIPTF

SEQ ID NO: 33 - protein, 16 aa, synthetic construct
  REGION 1..16: note = Synthetic
CAVEGLTTDSWGKFQF

SEQ ID NO: 34 - protein, 13 aa, synthetic construct
  REGION 1..13: note = Synthetic
CLVGSSNTGKLIF

SEQ ID NO: 35 - protein, 13 aa, synthetic construct
  REGION 1..13: note = Synthetic
CACPSGGYQKVTF

SEQ ID NO: 36 - protein, 14 aa, synthetic construct
  REGION 1..14: note = Synthetic
CAVRDEGSARQLTF

SEQ ID NO: 37 - protein, 18 aa, synthetic construct
  REGION 1..18: note = Synthetic
CALSVYNAGGTSYGKLTF

SEQ ID NO: 38 - protein, 12 aa, synthetic construct
  REGION 1..12: note = Synthetic
CAAWEGDDKIIF

SEQ ID NO: 39 - protein, 16 aa, synthetic construct
  REGION 1..16: note = Synthetic
CALSEAFTDSWGKFQF

SEQ ID NO: 40 - protein, 13 aa, synthetic construct
  REGION 1..13: note = Synthetic
CAGQKTSYDKVIF

SEQ ID NO: 41 - protein, 13 aa, synthetic construct
  REGION 1..13: note = Synthetic
CAARSGGYQKVTF

SEQ ID NO: 42 - protein, 13 aa, synthetic construct
  REGION 1..13: note = Synthetic
CAVSGASYDKVIF

SEQ ID NO: 43 - protein, 12 aa, synthetic construct
  REGION 1..12: note = Synthetic
CAGGGSQGNLIF

SEQ ID NO: 44 - protein, 12 aa, synthetic construct
  REGION 1..12: note = Synthetic
CAAVSNDYKLSF

SEQ ID NO: 45 - protein, 12 aa, synthetic construct
  REGION 1..12: note = Synthetic
CATDTHNNDMRF

SEQ ID NO: 46 - protein, 13 aa, synthetic construct
  REGION 1..13: note = Synthetic
CAVFSNSGYALNF

SEQ ID NO: 47 - protein, 13 aa, synthetic construct
  REGION 1..13: note = Synthetic
CALSEAYGQNFVF

SEQ ID NO: 48 - protein, 15 aa, synthetic construct
  REGION 1..15: note = Synthetic
CAVATINNAGNMLTF

SEQ ID NO: 49-50 - skipped (2 entries)

SEQ ID NO: 51 - protein, 13 aa, synthetic construct
  REGION 1..13: note = Synthetic
CATSDRGSPEAFF

SEQ ID NO: 52 - protein, 13 aa, synthetic construct
  REGION 1..13: note = Synthetic
CASSRTSNGEQFF

SEQ ID NO: 53 - protein, 12 aa, synthetic construct
  REGION 1..12: note = Synthetic
CASRPQGDGYTF

SEQ ID NO: 54 - protein, 16 aa, synthetic construct
  REGION 1..16: note = Synthetic
CASSYSLGEDGDTQYF

SEQ ID NO: 55 - protein, 11 aa, synthetic construct
  REGION 1..11: note = Synthetic
CASSLSYEQYF

SEQ ID NO: 56 - protein, 13 aa, synthetic construct
  REGION 1..13: note = Synthetic
CASSGSGTYEQYF

SEQ ID NO: 57 - protein, 16 aa, synthetic construct
  REGION 1..16: note = Synthetic
CASIPENSGNTGELFF

SEQ ID NO: 58 - protein, 14 aa, synthetic construct
  REGION 1..14: note = Synthetic
CASSYGGGTDTQYF

SEQ ID NO: 59 - protein, 14 aa, synthetic construct
  REGION 1..14: note = Synthetic
CASSLGQGYYGYTF

SEQ ID NO: 60 - protein, 17 aa, synthetic construct
  REGION 1..17: note = Synthetic
CASSLGPSSPSTDTQYF

SEQ ID NO: 61 - protein, 15 aa, synthetic construct
  REGION 1..15: note = Synthetic
CASSKSGQGSYEQYF

SEQ ID NO: 62 - protein, 15 aa, synthetic construct
  REGION 1..15: note = Synthetic
CSTPMWQGEGTEAFF

SEQ ID NO: 63 - protein, 15 aa, synthetic construct
  REGION 1..15: note = Synthetic
CASSLATTARYEQYF

SEQ ID NO: 64 - protein, 14 aa, synthetic construct
  REGION 1..14: note = Synthetic
CASSLGGYPNGYTF

SEQ ID NO: 65 - protein, 13 aa, synthetic construct
  REGION 1..13: note = Synthetic
CASSPGLGETQYF

SEQ ID NO: 66 - protein, 14 aa, synthetic construct
  REGION 1..14: note = Synthetic
CASSFGGTQETQYF

SEQ ID NO: 67 - protein, 12 aa, synthetic construct
  REGION 1..12: note = Synthetic
CAWSTHNVEQYF

SEQ ID NO: 68 - protein, 13 aa, synthetic construct
  REGION 1..13: note = Synthetic
CASSLTGSHEQYF

SEQ ID NO: 69 - protein, 13 aa, synthetic construct
  REGION 1..13: note = Synthetic
CASSSGQGQPQHF

SEQ ID NO: 70 - protein, 16 aa, synthetic construct
  REGION 1..16: note = Synthetic
CASSLDSRGGNTEAFF

SEQ ID NO: 71 - protein, 15 aa, synthetic construct
  REGION 1..15: note = Synthetic
CASSYLDSLNTEAFF

SEQ ID NO: 72 - protein, 13 aa, synthetic construct
  REGION 1..13: note = Synthetic
CASSYGPNYGYTF

SEQ ID NO: 73 - protein, 15 aa, synthetic construct
  REGION 1..15: note = Synthetic
CASSFTGHRTYEQYF

SEQ ID NO: 74 - protein, 16 aa, synthetic construct
  REGION 1..16: note = Synthetic
CASSFLDSPLNEKLFF

SEQ ID NO: 75 - protein, 18 aa, synthetic construct
  REGION 1..18: note = Synthetic
CASSSASTGTGFLETQYF

SEQ ID NO: 76 - protein, 14 aa, synthetic construct
  REGION 1..14: note = Synthetic
CASTVAGVGYEQYF

SEQ ID NO: 77 - protein, 13 aa, synthetic construct
  REGION 1..13: note = Synthetic
CAWSVGGIGGYTF

SEQ ID NO: 78 - protein, 19 aa, synthetic construct
  REGION 1..19: note = Synthetic
CASSLGLAGDDLSTDTQYF

SEQ ID NO: 79 - protein, 16 aa, synthetic construct
  REGION 1..16: note = Synthetic
CASSGRWQLAPTSSTS

SEQ ID NO: 80 - protein, 16 aa, synthetic construct
  REGION 1..16: note = Synthetic
CASSEAQRWGNTEAFF

SEQ ID NO: 81 - protein, 12 aa, synthetic construct
  REGION 1..12: note = Synthetic
CASSWGGGELFF

SEQ ID NO: 82 - protein, 13 aa, synthetic construct
  REGION 1..13: note = Synthetic
CASSGDSNTEAFF

SEQ ID NO: 83 - protein, 13 aa, synthetic construct
  REGION 1..13: note = Synthetic
CAASVKSNYQLIW

SEQ ID NO: 84 - protein, 13 aa, synthetic construct
  REGION 1..13: note = Synthetic
CASSLGRESGYTF

SEQ ID NO: 85 - protein, 14 aa, synthetic construct
  REGION 1..14: note = Synthetic
CASSYGRGTVPLHF

SEQ ID NO: 86 - protein, 15 aa, synthetic construct
  REGION 1..15: note = Synthetic
CASKQTSGRYNEQFF

SEQ ID NO: 87 - protein, 13 aa, synthetic construct
  REGION 1..13: note = Synthetic
CAWSVGGGTEAFF

SEQ ID NO: 88 - DNA, 28 bp, synthetic construct
  misc_feature 1..28: note = Synthetic
  misc_feature 1..2: note = n is a, c, g, or t
  source 1..28: mol_type = other DNA
nnaaaaaaaaaaaaaaaaaaaaaaaaaa

SEQ ID NO: 89 - DNA, 25 bp, synthetic construct
  misc_feature 1..25: note = Synthetic
  source 1..25: mol_type = other DNA
gtactctgcgttgataccactgatt

SEQ ID NO: 90 - DNA, 41 bp, synthetic construct
  misc_feature 1..41: note = Synthetic
  source 1..41: mol_type = other DNA
cccagatcggaagagcgtcgtgtactctgcgttgataccac

SEQ ID NO: 91 - DNA, 43 bp, synthetic construct
  misc_feature 1..43: note = Synthetic
  misc_feature 29..30: note = Site of Illumina i5 adapter index
  source 1..43: mol_type = other DNA
aatgatacggcgaccaccgagatctacactcgtcggcagcgtc

SEQ ID NO: 92 - DNA, 25 bp, synthetic construct
  misc_feature 1..25: note = Synthetic
  source 1..25: mol_type = other DNA
ttagtcaccatagttgcgtctcatg

SEQ ID NO: 93 - protein, 13 aa, synthetic construct
  REGION 1..13: note = Synthetic
CAVNGRRQAKSTF

SEQ ID NO: 94 - protein, 11 aa, synthetic construct
  REGION 1..11: note = Synthetic
CAFCGGNQFYF

SEQ ID NO: 95 - protein, 13 aa, synthetic construct
  REGION 1..13: note = Synthetic
CASSLGLGQPQHF

SEQ ID NO: 96 - DNA, 22 bp, synthetic construct
  misc_feature 1..22: note = Synthetic
  source 1..22: mol_type = other DNA
tacacgacgctcttccgatctg

SEQ ID NO: 97 - DNA, 20 bp, synthetic construct
  misc_feature 1..20: note = Synthetic
  source 1..20: mol_type = other DNA
acggcagggtcagggttctg

SEQ ID NO: 98 - DNA, 20 bp, synthetic construct
  misc_feature 1..20: note = Synthetic
  source 1..20: mol_type = other DNA
gcgacctcgggtgggaacac

SEQ ID NO: 99 - DNA, 20 bp, synthetic construct
  misc_feature 1..20: note = Synthetic
  source 1..20: mol_type = other DNA
cagaaccctgaccctgccgt

SEQ ID NO: 100 - DNA, 20 bp, synthetic construct
  misc_feature 1..20: note = Synthetic
  source 1..20: mol_type = other DNA
gtgttcccacccgaggtcgc

(19) United States
(12) Patent Application Publication
(10) Pub. No.: US 2023/0079640 A1
(43) Pub. Date: Mar. 16, 2023

(54) Methods of Amplifying Paired Transcript Sequences from Single Cells

(71) Applicant: Berkeley Lights, Inc., Emeryville, CA (US)

(72) Inventors: Duane Smith, Berkeley, CA (US); Jason M. McEwen, El Cerrito, CA (US)

(73) Assignee: Berkeley Lights, Inc., Emeryville, CA (US)

(21) Appl. No.: 17/931,217

(22) Filed: Sep. 12, 2022

Related U.S. Application Data
(63) Continuation of application No. PCT/US2021/022161, filed on Mar. 12, 2021.
(60) Provisional application No. 62/989,590, filed on Mar. 14, 2020.

(51) Int. Cl. C12Q 1/6806; C12Q 1/6865; C12Q 1/6876

(52) U.S. Cl. CPC C12Q 1/6806; C12Q 1/6865; C12Q 1/6876

(57) ABSTRACT

The disclosure provides methods for amplification of paired transcript sequences from a single cell, such as, but not limited to alpha and beta T cell receptor (TCR) sequences from a single T cell. The method can include: placing a single T cell into a cell lysis solution to provide a T cell lysate comprising RNA; generating first strand cDNA from the RNA; amplifying the first strand cDNA to provide amplified cDNA; and amplifying the alpha and beta TCR sequences from the amplified cDNA in a single reaction, wherein the alpha and beta TCR sequences are amplified using three sets of TCR amplification primers. In some instances, the methods can be performed using no more than six sets of primers.

### CROSS REFERENCE TO RELATED APPLICATIONS

This application is a continuation of International Application No. PCT/US2021/022161, filed Mar. 12, 2021, which claims the benefits of priority to U.S. Provisional Application No. 62/989,590, filed Mar. 14, 2020, the entire contents of which are incorporated by reference herein for any purpose.

### SEQUENCE LISTING

This application is filed with a Sequence Listing in electronic format. The Sequence Listing is provided as a file entitled “2022-08-25_01149-0019-00PCT_Seq_List_ST26” created on Aug. 25, 2022, which is 126,359 bytes in size. The information in the electronic format of the sequence listing is incorporated herein by reference in its entirety.

### INTRODUCTION AND SUMMARY

The present disclosures pertain generally to the field of nucleic acid sequencing, and more particularly to the sequencing of specific target transcripts, such as transcripts that encode T cell receptor alpha and beta peptides, from single cells.

Despite advances in single cell sequencing technology, it remains difficult to obtain sequence with a high rate of success from specific target transcripts that are not highly expressed. Because of all the processing steps involved in obtaining the sequence of target transcripts, and the low level of starting RNA obtained upon lysis of a single cell, inefficiencies and loss of material during the processing steps rapidly reduces the ability to obtain reliable sequencing results. This problem is compounded when the goal is to obtain sequence from each member of a set of two or more related transcripts.

Sequencing TCRs plays important roles in monitoring immune responses. TCR repertoire diversity may serve as a biomarker for cancer progression. TCR repertoire also can help characterize recovery of immune repertoire after transplant. Sequencing TCRs allows finding characteristic changes in TCR repertoire diversity associated with pathologies. In addition, identification of antigen-specific TCRs by TCR sequencing may be used in generating engineered T cells for adoptive cell therapy. It is, however, difficult to obtain from single cells gene sequences having variability within a region, such as, but not limited to, a 5′ region of alpha and beta TCR sequences.

The present disclosure addresses these problems in the context of simultaneously amplifying and sequencing the transcripts of T cell receptor (TCR) alpha and beta chain peptides. However, the disclosed methods can be readily extended to other sets of target transcripts.

In one aspect, the disclosure provides methods for paired amplification of alpha and beta T cell receptor (TCR) sequences from a single T cell. In certain embodiments, the method comprises: placing a single T cell into a cell lysis solution to provide a T cell lysate comprising RNA; generating first strand cDNA from the RNA; amplifying the first strand cDNA to provide amplified cDNA; and amplifying the alpha and beta TCR sequences from the amplified cDNA in a single reaction, wherein the alpha and beta TCR sequences are amplified using three sets of TCR amplification primers. In certain embodiments, the method is performed using no more than six sets of primers. In certain embodiments, the methods further comprise sequencing the amplified alpha and beta TCR sequences (e.g., using a next generation sequencing methodology). In certain related embodiments, the methods further comprise further amplifying the alpha and beta TCR sequences for the purpose of indexing the sequences.

In other aspects, the disclosure provides a kit for use in methods of amplifying paired transcript sequences from a single cell such as any of those disclosed herein.

Applicant has surprisingly discovered that a simplified set of primers can successfully amplify both gene forms of beta TCR sequences as well as alpha TCR sequences in the same amplification reaction. Surprisingly, beta1 and beta2 sequences can be amplified with the same 3′ primer. This was not known in the art, and there was no reasonable expectation that these two sequences could be amplified using the same 3′ primer. Thus, all of the TCR alpha, beta1 and beta2 sequences can be amplified using only three primers in total, a single 5′ primer and two 3′ primers. This provides a significant improvement over methods currently available.

Additionally, the methods disclosed herein may provide for full length cDNA, so the total transcriptome can be interrogated as desired. The methods disclosed herein may permit returning to the full-length amplicons to look at other genes, other splice variants and the like. Splice variants can be interrogated to explore what it particularly affects. Therefore, a variety of pathways can be investigated from the cDNA captured from one cell.

Additionally, the methods described herein may further provide for chain of custody from a particular single cell. In an optional aspect of the methods, cells may be provided from a microfluidic device, where the cells may have been enriched, stimulated, and/or selected for desirable phenotypes or production of biological products of interest. The information about such enrichment, stimulation, phenotype, or production can be correlated to identify precisely the cell (and its source location within the microfluidic device) and the nucleic acid sequences obtained from the cell.

The following numbered embodiments are provided herein.
Embodiment 1. A method for paired amplification of alpha and beta T cell receptor (TCR) sequences from a single T cell, the method comprising:
   placing a single T cell into a cell lysis solution to provide a T cell lysate comprising RNA;
   generating first strand cDNA from the RNA;
   amplifying the first strand cDNA to provide amplified cDNA; and
   amplifying the alpha and beta TCR sequences from the amplified cDNA in a single reaction, wherein the alpha and beta TCR sequences are amplified using three sets of TCR amplification primers.
Embodiment 2. The method of embodiment 1, wherein generating first strand cDNA comprises: performing a reverse transcription (RT) reaction by contacting the RNA with a first RT primer set, a second RT primer set, and an enzyme having reverse transcriptase activity, for a first period of time and under conditions that generate first strand cDNA.
Embodiment 3. The method of embodiment 2, wherein the RNA is contacted with the first RT primer set and the enzyme having reverse transcriptase activity prior to being contacted with the second RT primer set.
Embodiment 4. The method of embodiment 3, wherein the RNA is maintained at a temperature of about 10° C. or less (e.g., about 9° C., about 8° C., about 7° C., about 6° C., about 5° C., about 4° C., or less) prior to being contacted with the enzyme having reverse transcriptase activity.
Embodiment 5. The method of any one of embodiments 2 to 4, wherein the first RT primer set comprises one or more first RT primers, each first RT primer comprising a poly-T subsequence.
Embodiment 6. The method of embodiment 5, wherein the poly-T subsequence of each first RT primer of the first RT primer set includes a string of at least 20 thymidine nucleotides (e.g., deoxyribonucleotides).
Embodiment 7. The method of embodiment 5 or 6, wherein each first RT primer of the first RT primer set comprises a 3′ terminal VN subsequence, wherein V is any nucleotide selected from A, C, and G, wherein N is any nucleotide selected from A, C, G, and T, and wherein the poly-T subsequence is located 5′ to the 3′ terminal VN subsequence.
Embodiment 8. The method of any one of embodiments 2 to 7, wherein each first RT primer of the first RT primer set comprises a first amplification priming subsequence.
Embodiment 9. The method of embodiment 8, wherein the first amplification priming subsequence is located 5′ to a/the poly-T subsequence.
Embodiment 10. The method of any one of embodiments 2 to 9, wherein the second RT primer set comprises one or more template switching oligos (TSOs).
Embodiment 11. The method of embodiment 10, wherein the second RT primer set comprises a single TSO.
Embodiment 12. The method of embodiment 10 or 11, wherein each TSO of the second RT primer set comprises a 3′ terminal ribonucleotide subsequence.
Embodiment 13. The method of embodiment 12, wherein the 3′ terminal ribonucleotide subsequence comprises a string of at least three ribonucleotides.
Embodiment 14. The method of embodiment 12 or 13, wherein the 3′ terminal ribonucleotide subsequence comprises a rGrGrG sequence.
Embodiment 15. The method of any one of embodiments 10 to 14, wherein each TSO of the second RT primer set comprises a second amplification priming subsequence located 5′ to the 3′ terminal ribonucleotide subsequence.
Embodiment 16. The method of embodiment 15, wherein the first amplification priming subsequence of the first RT primer set and the second amplification priming subsequence of the second RT primer set have substantially identical nucleotide sequences.
Embodiment 17. The method of any one of embodiments 10 to 16, wherein each TSO of the second RT primer set comprises a barcode subsequence.
Embodiment 18. The method of embodiment 17, wherein the barcode subsequence is located 5′ to a/the terminal ribonucleotide subsequence and 3′ to a/the second amplification priming subsequence.
Embodiment 19. The method of any one of embodiments 1 to 18, wherein amplifying the first strand cDNA comprises contacting the first strand cDNA with a third primer set comprising one or more third primers, for a second period of time and under conditions that generate amplified cDNA.
Embodiment 20. The method of embodiment 19, wherein the third primer set comprises a single primer.
Embodiment 21. The method of embodiment 19 or 20, wherein each third primer of the third primer set comprises a third amplification priming subsequence.
Embodiment 22. The method of embodiment 21, wherein the first amplification priming subsequence of the first RT primer set, the second amplification priming subsequence of the second RT primer set, and the third amplification priming subsequence of the third primer set all have substantially identical nucleotide sequences.
Embodiment 23. The method of any one of embodiments 1 to 22, wherein generating first strand cDNA and amplifying the first strand cDNA are performed without an intervening purification of the first strand cDNA.
Embodiment 24. The method of any one of embodiments 1 to 23, wherein amplifying the alpha and beta TCR sequences comprises contacting the amplified cDNA with the three sets of TCR amplification primers for a third period of time and under conditions that provide amplification of paired alpha and beta T cell receptor (TCR) sequences from the amplified cDNA, wherein the three sets of TCR amplification primers comprise a first set of TCR amplification primers, a set of TCR alpha chain amplification primers, and a set of TCR beta chain amplification primers.
Embodiment 25. The method of embodiment 24, wherein each primer of the set of TCR alpha chain amplification primers comprises a TCR alpha chain complement subsequence that is substantially identical to a complement sequence of a constant region sequence of a TCR alpha chain.
Embodiment 26. The method of embodiment 25, wherein the constant region sequence of the TCR alpha chain is located at or proximal to a junction with a variable region sequence of the TCR alpha chain.
Embodiment 27. The method of embodiment 25 or 26, wherein each primer of the set of TCR alpha chain amplification primers further comprises a fourth amplification priming subsequence located 5′ to the TCR alpha chain complement subsequence.
Embodiment 28. The method of any one of embodiments 25 to 27, wherein each primer of the set of TCR alpha chain amplification primers further comprises a barcode subsequence (e.g., a string of N nucleotides) and/or a unique molecular identifier (UMI) subsequence.
Embodiment 29. The method of any one of embodiments 24 to 28, wherein the set of TCR alpha chain amplification primers comprises a single TCR alpha chain amplification primer.
Embodiment 30. The method of any one of embodiments 24 to 29, wherein each primer of the set of TCR beta chain amplification primers comprises a TCR beta chain complement subsequence that is substantially identical to a complement sequence of a constant region sequence of a TCR beta chain.
Embodiment 31. The method of embodiment 30, wherein the constant region sequence of the TCR beta chain is located at or proximal to a junction with a variable region sequence of the TCR beta chain.
Embodiment 32. The method of embodiment 30 or 31, wherein each primer of the set of TCR beta chain amplification primers further comprises a fifth amplification priming subsequence located 5′ to the TCR beta chain complement subsequence.
Embodiment 33. The method of any one of embodiments 30 to 32, wherein each primer of the set of TCR beta chain amplification primers further comprises a barcode subsequence and/or a unique molecular identifier (UMI) subsequence.
Embodiment 34. The method of any one of embodiments 24 to 33, wherein the set of TCR beta chain amplification primers comprises a single TCR beta chain amplification primer.
Embodiment 35. The method of embodiment 34, wherein amplifying the beta TCR sequences comprises amplifying both beta chain 1 sequence and beta chain 2 sequence using the single TCR beta chain amplification primer.
Embodiment 36. The method of embodiment 35, wherein the single TCR beta chain amplification primer comprises SEQ ID NO: 6.
Embodiment 37. The method of any one of embodiments 24 to 36, wherein each primer of the first set of TCR amplification primers comprises an anchor subsequence that is substantially identical to at least a subsequence of each TSO of the second RT primer set.
Embodiment 38. The method of any one of embodiments 24 to 37, wherein a 3′ terminal subsequence of each primer of the first set of TCR amplification primers comprises a TG-3′ or a TGG-3′ sequence.
Embodiment 39. The method of embodiment 37 or 38, wherein each primer of the first set of TCR amplification primers further comprises a sixth amplification priming subsequence located 5′ to the anchor subsequence.
Embodiment 40. The method of any one of embodiments 37-39, wherein each primer of the first set of TCR amplification primers further comprises a barcode subsequence and/or a unique molecular identifier subsequence.
Embodiment 41. The method of any one of embodiments 24 to 40, wherein the first set of TCR amplification primers comprises a single TCR amplification primer.
Embodiment 42. The method of any one of embodiments 24 to41, wherein the fourth amplification priming subsequence of the TCR alpha chain amplification primer set, the fifth amplification priming subsequence of the TCR beta chain amplification primer set, and the sixth amplification priming subsequence of the first TCR amplification primer set all share a substantially identical sequence.
Embodiment 43. The method of any one of embodiments 24 to 42, wherein amplifying the alpha and beta TCR sequences comprises using the three sets of TCR amplification primers in a molar ratio of 2:1:1 of the first set of TCR amplification primers:the set of TCR alpha chain amplification primers:the set of TCR beta chain amplification primers.
Embodiment 44. The method of any one of embodiments 24 to 42, wherein amplifying the alpha and beta TCR sequences comprises using the three sets of TCR amplification primers in a ratio of 2:1:1 of a weight of the first set of TCR amplification primers:a weight of the set of TCR alpha chain amplification primers:a weight of the set of TCR beta chain amplification primers.
Embodiment 45. The method of any one of embodiments 24 to 44, wherein each primer of the first set of TCR amplification primers comprises SEQ ID NO: 96, each primer of the set of TCR alpha chain amplification primers comprises SEQ ID NO: 97 and each primer of the set of TCR beta chain amplification primers comprises SEQ ID NO: 98.
Embodiment 46. The method of any one of embodiments 1 to 45, wherein the method is performed using no more than six sets of primers.
Embodiment 47. The method of any one of embodiments 24 to 46, wherein amplifying the alpha and beta TCR sequences comprises amplifying the alpha TCR sequences, beta chain 1 sequence, and beta chain 2 sequence using the single TCR alpha chain amplification primer, the single beta chain amplification primer, and the single TCR amplification primer.
Embodiment 48. The method of any one of embodiments 1 to 47, wherein placing the single T cell into the cell lysis solution comprises exporting the single T cell from a microfluidic chip.
Embodiment 49. The method of any one of embodiments 1 to 47, wherein placing the single T cell into the cell lysis solution comprises depositing the single T cell into the lysis solution using FACS.
Embodiment 50. The method of any one of embodiments 1 to 49, further comprising, before placing the T cell in the lysis solution, selecting the T cell based on cell-surface expression of a marker selected from CD3, CD4, CD8, CD45RO, CD45RA, CCR7, CD62L, PD-1, CD137, or any combination thereof.
Embodiment 51. The method of embodiment 50, wherein selecting the T cell comprises performing a positive selection.
Embodiment 52. The method of embodiment 50 or 51, wherein selecting the T cell comprises performing a negative selection.
Embodiment 53. The method of any one of embodiments 1 to 52 further comprising, before placing the T cell in the lysis solution, selecting the T cell based on expression by the T cell of one or more cytokines.
Embodiment 54. The method of embodiment 53, wherein the one or more cytokines is selected from IFNgamma, TNFalpha, IL2, or a combination thereof.
Embodiment 55. The method of embodiment 53 or 54, wherein the single T cell is selected based on being positive for cytokine secretion.
Embodiment 56. The method of any one of embodiments 1 to 55 further comprising purifying the RNA from the T cell lysate.
Embodiment 57. The method of embodiment 56, wherein the RNA is purified using a bead-based purification method.
Embodiment 58. The method of embodiment 57, wherein generating first strand cDNA is performed in the presence of beads used in the bead-based purification method.
Embodiment 59. The method of embodiment 58, wherein amplifying the first strand cDNA is performed in the presence of beads used in the bead-based purification method.
Embodiment 60. The method of any one of embodiments 1 to 59 further comprising purifying the amplified first strand cDNA.
Embodiment 61. The method of any one of embodiments 1 to 60 further comprising further amplifying the alpha and beta TCR sequences to add index sequences for sequencing.
Embodiment 62. The method of any one of embodiments 1 to 61 further comprising performing the method on a plurality of single T cells.
Embodiment 63. The method of embodiment 62, wherein paired alpha and beta T cell receptor (TCR) sequences are obtained from at least 50% of the plurality of single T cells.
Embodiment 64. A kit for use in the methods according to any one of embodiments 1-63.
Embodiment 65. A kit comprising:
   a first RT primer set comprising one or more first RT primers;
   a second RT primer set comprising one or more second RT primers; and
   a third RT primer set comprising one or more third RT primers.
Embodiment 66. The kit of embodiment 65, further comprising:
   a first set of TCR amplification primers;
   a set of TCR alpha chain amplification primers; and
   a set of TCR beta chain amplification primers.
Embodiment 67. The kit of embodiment 66, wherein the set of TCR beta chain amplification primers has a single forward amplification primer and a single reverse amplification primer.
Embodiment 68. The kit of embodiment 66 or 67, further comprising one or more of: dNTP Mix, RT Buffer, RT Enzyme, RT Additive, RNase inhibitor, PCR enzyme K, in any combination thereof.
Embodiment 69. The kit according to embodiment 66-68, wherein each primer of the first set of TCR amplification primers comprises SEQ ID NO: 96, each primer of the set of TCR alpha chain amplification primers comprises SEQ ID NO: 97, and each primer of the set of TCR beta chain amplification primers comprises SEQ ID NO: 98.

Additional embodiments are provided herein:
Embodiment A1. A method for paired amplification of alpha and beta T cell receptor (TCR) sequences from a single T cell, the method comprising:
   placing a single T cell into a cell lysis solution to provide a T cell lysate comprising RNA;
   generating first strand cDNA from the RNA;
   amplifying the first strand cDNA to provide amplified cDNA; and
   amplifying the alpha and beta TCR sequences from the amplified cDNA in a single reaction,
   wherein the alpha and beta TCR sequences are amplified using three sets of TCR amplification primers.
Embodiment A2. The method of embodiment A1, wherein generating first strand cDNA comprises: performing a reverse transcription (RT) reaction by contacting the RNA with a first RT primer set, a second RT primer set, and an enzyme having reverse transcriptase activity, for a first period of time and under conditions sufficient to generate first strand cDNA.
Embodiment A3. The method of embodiment A2, wherein the RNA is contacted with the first RT primer set and the enzyme having reverse transcriptase activity prior to being contacted with the second RT primer.
Embodiment A4. The method of embodiment A3, wherein the RNA is maintained at a temperature of about 10° C. or less (e.g., about 9° C., about 8° C., about 7° C., about 6° C., about 5° C., about 4° C., or less) prior to being contacted with the enzyme having reverse transcriptase activity.
Embodiment A5. The method of embodiments A2 to A4, wherein the first RT primer set comprises one or more first RT primers, each first RT primer comprising a poly-T subsequence.
Embodiment A6. The method of embodiment A5, wherein the poly-T subsequence of each first RT primer of the first RT primer set includes a string of at least 20 thymidine nucleotides (e.g., deoxyribonucleotides). In some embodiments, the poly-T subsequence comprises a string of at least 25, 26, 27, 28, 29, 30, or more thymidine nucleotides.
Embodiment A7. The method of embodiments A5 or A6, wherein each first RT primer of the first RT primer set comprises a 3′ terminal VN subsequence, wherein V is any nucleotide (e.g., deoxyribonucleotide) selected from A, C, and G, wherein N is any nucleotide (e.g. deoxyribonucleotide) selected from A, C, G, and T, and wherein the poly-T subsequence is located 5′ to the 3′ terminal VN subsequence.
Embodiment A8. The method of any one of embodiments A2 to A7, wherein each first RT primer of the first RT primer set comprises a first amplification priming subsequence.
Embodiment A9. The method of embodiment A8, wherein the first amplification priming subsequence is located 5′ to a/the poly-T subsequence.
Embodiment A10. The method of any one of embodiments A2 to A9, wherein the second RT primer set comprises one or more template switching oligos (TSOs).
Embodiment A11. The method of embodiment A10, wherein the second RT primer set comprises a single TSO.
Embodiment A12. The method of embodiment A10 or A11, wherein each TSO of the second RT primer set comprises a 3′ terminal ribonucleotide subsequence.
Embodiment A13. The method of embodiment A12, wherein the 3′ terminal ribonucleotide subsequence comprises a string of at least three ribonucleotides.
Embodiment A14. The method of embodiment A12 or A13, wherein the 3′ terminal ribonucleotide subsequence comprises a rGrGrG sequence.
Embodiment A15. The method of any one of embodiments A10 to A14, wherein each TSO of the second RT primer set comprises a second amplification priming subsequence located 5′ to the 3′ terminal ribonucleotide subsequence.
Embodiment A16. The method of embodiment A15, wherein the first amplification priming subsequence of the first RT primer set and the second amplification priming subsequence of the second RT primer set have substantially identical nucleotide sequences.
Embodiment A17. The method of any one of embodiments A10 to A16, wherein each TSO of the second RT primer set comprises a barcode subsequence (e.g., a string of N nucleotides (e.g., 2 to 10 nucleotides, or 3 to 6 nucleotides), wherein each N in the string is any nucleotide (e.g. deoxyribonucleotide) selected from A, C, G, and T).
Embodiment A18. The method of embodiment A17, wherein the barcode subsequence is located 5′ to a/the terminal ribonucleotide subsequence and 3′ to a/the second amplification priming subsequence.
Embodiment A19. The method of any one of embodiments A1 to A18, wherein amplifying the first strand cDNA comprises contacting the first strand cDNA with a third primer set comprising one or more third primers, for a second period of time and under conditions sufficient to generate amplified cDNA.
Embodiment A20. The method of embodiments A19, wherein the third primer set comprises a single primer.
Embodiment A21. The method of embodiments A19 or A20, wherein each third primer of the third primer set comprises a third amplification priming subsequence.
Embodiment A22. The method of embodiment A21, wherein the first amplification priming subsequence of the first RT primer set, the second amplification priming subsequence of the second RT primer set, and the third amplification priming subsequence of the third primer set all have substantially identical nucleotide sequences.
Embodiment A23. The method of any one of embodiments A1 to A22, wherein generating first strand cDNA and amplifying the first strand cDNA are performed without an intervening purification of the first strand cDNA.
Embodiment A24. The method of any one of embodiments A1 to A23, wherein amplifying the alpha and beta TCR sequences comprises contacting the amplified cDNA with the three sets of TCR amplification primers for a third period of time and under conditions sufficient to provide amplification of paired alpha and beta T cell receptor (TCR) sequences from the amplified cDNA, wherein the three sets of TCR amplification primers comprise a first set of TCR amplification primers, a set of TCR alpha chain amplification primers, and a set of TCR beta chain amplification primers.
Embodiment A25. The method of embodiment A24, wherein each primer of the set of TCR alpha chain amplification primers comprises a TCR alpha chain complement subsequence that is substantially identical to a complement sequence of a constant region sequence of a TCR alpha chain.
Embodiment A26. The method of embodiment A25, wherein the constant region sequence of the TCR alpha chain is located at or proximal to a junction with a variable region sequence of the TCR alpha chain. (This can include having the primer sequence be a sequence that includes identity on both sides of the junction, or just on the constant region side of the junction.)
Embodiment A27. The method of embodiment A25 or A26, wherein each primer of the set of TCR alpha chain amplification primers further comprises a fourth amplification priming subsequence located 5′ to the TCR alpha chain complement subsequence.
Embodiment A28. The method of any one of embodiment A25 to A27, wherein each primer of the set of TCR alpha chain amplification primers further comprises a barcode subsequence (e.g., a string of N nucleotides (e.g., 2 to 10 nucleotides, or 3 to 6 nucleotides) and/or a unique molecular identifier (UMI) subsequence (e.g., a string of N nucleotides (e.g., 2 to 10 nucleotides, or 3 to 6 nucleotides), wherein each N in the string is any nucleotide (e.g. deoxyribonucleotide) selected from A, C, G, and T).
Embodiment A29. The method of any one of embodiments A24 to A28, wherein the set of TCR alpha chain amplification primers comprises a single TCR alpha chain amplification primer.
Embodiment A30. The method of any one of embodiments A 24 to A29, wherein each primer of the set of TCR beta chain amplification primers comprises a TCR beta chain complement subsequence that is substantially identical to a complement sequence of a constant region sequence of a TCR beta chain.
Embodiment A31. The method of embodiment A30, wherein the constant region sequence of the TCR beta chain is located at or proximal to a junction with a variable region sequence of the TCR beta chain.
Embodiment A32. The method of embodiment A30 or A31, wherein each primer of the set of TCR beta chain amplification primers further comprises a fifth amplification priming subsequence located 5′ to the TCR beta chain complement subsequence.
Embodiment A33. The method of any one of embodiments A30 to A32, wherein each primer of the set of TCR beta chain amplification primers further comprises a barcode subsequence (e.g., a string of N nucleotides (e.g., 2 to 10 nucleotides, or 3 to 6 nucleotides) and/or a unique molecular identifier (UMI) subsequence (e.g., a string of N nucleotides (e.g., 2 to 10 nucleotides, or 3 to 6 nucleotides), wherein each N in the string is any nucleotide (e.g. deoxyribonucleotide) selected from A, C, G, and T).
Embodiment A34. The method of any one of embodiments A24 to A33, wherein the set of TCR beta chain amplification primers comprises a single TCR beta chain amplification primer.
Embodiment A35. The method of any one of embodiments A24 to A34, wherein each primer of the first set of TCR amplification primers comprises an anchor subsequence that is substantially identical to at least a subsequence of each TSO of the second RT primer set.
Embodiment A36. The method of any one of embodiments A24 to A35, wherein a 3′ terminal subsequence of each primer of the first set of TCR amplification primers comprises a TG-3′ or a TGG-3′ sequence.
Embodiment A37. The method of embodiment A35 or A36, wherein each primer of the first set of TCR amplification primers further comprises a sixth amplification priming subsequence located 5′ to the anchor subsequence.
Embodiment A38. The method of any one of embodiments A35 to A37, wherein each primer of the first set of TCR amplification primers further comprises a barcode subsequence (e.g., a string of N nucleotides (e.g., 2 to 10 nucleotides, or 3 to 6 nucleotides) and/or a unique molecular identifier subsequence (e.g., a string of N nucleotides (e.g., 2 to 10 nucleotides, or 3 to 6 nucleotides), wherein each N in the string is any nucleotide (e.g. deoxyribonucleotide) selected from A, C, G, and T).
Embodiment A39. The method of any one of embodiments A24 to A38, wherein the first set of TCR amplification primers comprises a single TCR amplification primer.
Embodiment A40. The method of any one of embodiments A24 to A39, wherein the fourth amplification priming subsequence of the TCR alpha chain amplification primer set, the fifth amplification priming subsequence of the TCR beta chain amplification primer set, and the sixth amplification priming subsequence of the first TCR amplification primer set all share a substantially identical sequence.
Embodiment A41. The method of any one of embodiments A24 to A40, wherein amplifying the alpha and beta TCR sequences comprises using the three sets of TCR amplification primers in a molar ratio of 2:1:1 of the first set of TCR amplification primers:the set of TCR alpha chain amplification primers:the set of TCR beta chain amplification primers.
Embodiment A42. The method of any one of embodiments A24 to A40, wherein amplifying the alpha and beta TCR sequences comprises using the three sets of TCR amplification primers in a ratio of 2:1:1 of a weight of the first set of TCR amplification primers:a weight of the set of TCR alpha chain amplification primers:a weight of the set of TCR beta chain amplification primers.
Embodiment A43. The method of any one of embodiments A1 to A42, wherein the method is performed using no more than six sets of primers.
Embodiment A44. The method of any one of embodiments A1 to A43, wherein placing the single T cell into the cell lysis solution comprises exporting the single T cell from a microfluidic chip.
Embodiment A45. The method of any one of embodiments A1 to A44, wherein placing the single T cell into the cell lysis solution comprises depositing the single T cell into the lysis solution using FACS.
Embodiment A46. The method of any one of embodiments A1 to A45, further comprising, before placing the T cell in the lysis solution, selecting the T cell based on cell-surface expression of a marker selected from CD3, CD4, CD8, CD45RO, CD45RA, CCR7, CD62L, PD-1, CD137, or any combination thereof.
Embodiment A47. The method of embodiment A46, wherein selecting the T cell comprises performing a positive selection.
Embodiment A48. The method of embodiment A46 or A47, wherein selecting the T cell comprises performing a negative selection.
Embodiment A49. The method of any one of embodiments A1 to A47 further comprising, before placing the T cell in the lysis solution, selecting the T cell based on expression by the T cell of one or more cytokines.
Embodiment A50. The method of embodiment A49, wherein the one or more cytokines is selected from IFNgamma, TNFalpha, IL2, or a combination thereof.
Embodiment A51. The method of embodiment A49 or A50, wherein the single T cell is selected based on being positive for cytokine secretion.
Embodiment A52. The method of any one of embodiments A1 to A51 further comprising purifying the RNA from the T cell lysate.
Embodiment A53. The method of embodiment A52, wherein the RNA is purified using a bead-based purification method.
Embodiment A54. The method of embodiment A53, wherein generating first strand cDNA is performed in the presence of beads used in the bead-based purification method.
Embodiment A55. The method of embodiment A54, wherein amplifying the first strand cDNA is performed in the presence of beads used in the bead-based purification method.
Embodiment A56. The method of any one of embodiments A1 to A55 further comprising purifying the amplified first strand cDNA. (e.g., using a bead-based purification method).
Embodiment A57. The method of any one of embodiments A1 to A56 further comprising further amplifying the alpha and beta TCR sequences to add index sequences (e.g., to facilitate next generation sequencing (NGS)).
Embodiment A58. The method of any one of embodiments A1 to A57 further comprising performing the method on a plurality (e.g., 2, 3, 4, 5, 6, 7, 8, 9, 10, 15, 20, 25, 30, 40, 48, 50, 60, 70, 80, 90, 96, or more) of single T cells.
Embodiment A59. The method of embodiment A58, wherein paired alpha and beta T cell receptor (TCR) sequences are obtained from at least 50% (e.g., at least 55%, 60%, 65%, 70%, 75%, 80%, 85%, 90%, or more) of the plurality of single T cells.

Additional embodiments are provided herein.
Embodiment B1. A method for paired amplification of predetermined paired sequences of interest from a single cell, the method comprising:
   placing a single cell into a cell lysis solution to provide a cell lysate comprising RNA;
   generating first strand cDNA from the RNA;
   amplifying the first strand cDNA to provide amplified cDNA; and
   amplifying the predetermined paired sequences of interest from the amplified cDNA in a single reaction, wherein the paired sequences are amplified using three sets of gene specific amplification primers. More generally, for n paired (or grouped) sequences, n+1 or fewer (e.g., n) gene specific amplification primer sets can be used to amplify the paired sequences.
Embodiment B2. The method of embodiment B1, wherein amplifying the predetermined paired sequences of interest comprises amplifying two or more homologous sequences, two or more variant sequences, or two or more sequences that encode peptides that form a complex with one another.
Embodiment B3. The method of embodiment B1 or B2, wherein generating first strand cDNA comprises: performing a reverse transcription (RT) reaction by contacting the RNA with a first RT primer set, a second RT primer set, and an enzyme having reverse transcriptase activity, for a first period of time and under conditions that generate first strand cDNA.
Embodiment B4. The method of embodiment B3, wherein the RNA is contacted with the first RT primer set and the enzyme having reverse transcriptase activity prior to being contacted with the second RT primer set.
Embodiment B5. The method of embodiment B4, wherein the RNA is maintained at a temperature of about 10° C. or less (e.g., about 9° C., about 8° C., about 7° C., about 6° C., about 5° C., about 4° C., or less) prior to being contacted with the enzyme having reverse transcriptase activity.
Embodiment B6. The method of any one of embodiments B3 to B5, wherein the first RT primer set comprises one or more first RT primers, each first RT primer comprising a poly-T subsequence.
Embodiment B7. The method of embodiment B6, wherein the poly-T subsequence of each first RT primer of the first RT primer set includes a string of at least 20 thymidine nucleotides (e.g., deoxyribonucleotides).
Embodiment B8. The method of embodiment B6 or 7, wherein each first RT primer of the first RT primer set comprises a 3′ terminal VN subsequence, wherein V is any nucleotide selected from A, C, and G, wherein N is any nucleotide selected from A, C, G, and T, and wherein the poly-T subsequence is located 5′ to the 3′ terminal VN subsequence.
Embodiment B9. The method of any one of embodiments B3 to B8, wherein each first RT primer of the first RT primer set comprises a first amplification priming subsequence.
Embodiment B10. The method of embodiment B9, wherein the first amplification priming subsequence is located 5′ to a/the poly-T subsequence.
Embodiment B11. The method of any one of embodiments B3 to B10, wherein the second RT primer set comprises one or more template switching oligos (TSOs).
Embodiment B12. The method of embodiment B11, wherein the second RT primer set comprises a single TSO.
Embodiment B13. The method of embodiment B11 or B12, wherein each TSO of the second RT primer set comprises a 3′ terminal ribonucleotide subsequence.
Embodiment B14. The method of embodiment B13, wherein the 3′ terminal ribonucleotide subsequence comprises a string of at least three ribonucleotides.
Embodiment B15. The method of embodiment B13 or B14, wherein the 3′ terminal ribonucleotide subsequence comprises a rGrGrG sequence.
Embodiment B16. The method of any one of embodiments B11 to B15, wherein each TSO of the second RT primer set comprises a second amplification priming subsequence located 5′ to the 3′ terminal ribonucleotide subsequence.
Embodiment B17. The method of embodiment B16, wherein the first amplification priming subsequence of the first RT primer set and the second amplification priming subsequence of the second RT primer set have substantially identical nucleotide sequences.
Embodiment B18. The method of any one of embodiments B11 to B17, wherein each TSO of the second RT primer set comprises a barcode subsequence.
Embodiment B19. The method of embodiment B18, wherein the barcode subsequence is located 5′ to a/the terminal ribonucleotide subsequence and 3′ to a/the second amplification priming subsequence.
Embodiment B20. The method of any one of embodiments B1 to B19, wherein amplifying the first strand cDNA comprises contacting the first strand cDNA with a third primer set comprising one or more third primers, for a second period of time and under conditions that generate amplified cDNA.
Embodiment B21. The method of embodiment B20, wherein the third primer set comprises a single primer.
Embodiment B22. The method of embodiment B20 or B21, wherein each third primer of the third primer set comprises a third amplification priming subsequence.
Embodiment B23. The method of embodiment B22, wherein the first amplification priming subsequence of the first RT primer set, the second amplification priming subsequence of the second RT primer set, and the third amplification priming subsequence of the third primer set all have substantially identical nucleotide sequences.
Embodiment B24. The method of any one of embodiments B1 to B23, wherein generating first strand cDNA and amplifying the first strand cDNA are performed without an intervening purification of the first strand cDNA.
Embodiment B25. The method of any one of embodiments B1 to B24, wherein amplifying the predetermined paired sequences of interest comprising a first gene specific sequence, a second gene specific sequence, optionally a third gene specific sequence, comprises contacting the amplified cDNA with the three sets of gene specific amplification primers for a third period of time and under conditions that provide amplification of the predetermined paired sequences of interest from the amplified cDNA, wherein the three sets of gene specific amplification primers comprise a first set of shared sequence amplification primers, a set of first gene sequence amplification primers, and a set of second gene sequence amplification primers.
Embodiment B26. The method of embodiment B25, wherein each primer of the set of first gene sequence amplification primers comprises a first gene specific complement subsequence that is substantially identical to a complement sequence of a constant region sequence of a first gene specific sequence.
Embodiment B27. The method of embodiment B26, wherein the constant region sequence of the first gene specific sequence is located at or proximal to a junction with a variable region sequence of the first gene specific sequence.
Embodiment B28. The method of embodiment B26 or B27, wherein each primer of the set of first gene sequence amplification primers further comprises a fourth amplification priming subsequence located 5′ to the first gene specific complement subsequence.
Embodiment B29. The method of any one of embodiments B25 to B28, wherein each primer of the set of first gene sequence amplification primers further comprises a barcode subsequence (e.g., a string of N nucleotides) and/or a unique molecular identifier (UMI) subsequence.
Embodiment B30. The method of any one of embodiments B25 to B29, wherein the set of first gene sequence amplification primers comprises a single first gene sequence amplification primer.
Embodiment B31. The method of any one of embodiments B25 to B30, wherein each primer of the set of second gene sequence amplification primers comprises a second gene specific complement subsequence that is substantially identical to a complement sequence of a constant region sequence of a second gene specific sequence.
Embodiment B32. The method of embodiment B31, wherein the constant region sequence of the second gene specific sequence is located at or proximal to a junction with a variable region sequence of the second gene specific sequence.
Embodiment B33. The method of embodiment B31 or B32, wherein each primer of the set of second gene sequence amplification primers further comprises a fifth amplification priming subsequence located 5′ to the second gene specific complement subsequence.
Embodiment B34. The method of any one of embodiments B31 to B33, wherein each primer of the set of second gene sequence amplification primers further comprises a barcode subsequence and/or a unique molecular identifier (UMI) subsequence.
Embodiment B35. The method of any one of embodiments B25 to B34, wherein the set of second gene sequence amplification primers comprises a single second gene sequence amplification primer.
Embodiment B36. The method of embodiment B35, wherein amplifying the predetermined paired sequences of interest comprises amplifying both the second gene specific sequence and the third gene specific sequence using the single second gene sequence amplification primer.
Embodiment B37. The method of any one of embodiments B25 to B36, wherein each primer of the first set of shared sequence amplification primers comprises an anchor subsequence that is substantially identical to at least a subsequence of each TSO of the second RT primer set.
Embodiment B38. The method of any one of embodiments B25 to B37, wherein a 3′ terminal subsequence of each primer of the first set of shared sequence amplification primers comprises a TG-3′ or a TGG-3′ sequence.
Embodiment B39. The method of embodiment B37 or B38, wherein each primer of the first set of shared sequence amplification primers further comprises a sixth amplification priming subsequence located 5′ to the anchor subsequence.
Embodiment B40. The method of any one of embodiments B25 to B39, wherein each primer of the first set of shared sequence amplification primers further comprises a barcode subsequence and/or a unique molecular identifier subsequence.
Embodiment B41. The method of any one of embodiments B24 to B40, wherein the first set of shared sequence amplification primers comprises a single shared sequence amplification primer.
Embodiment B42. The method of any one of embodiments B24 to B41, wherein the fourth amplification priming subsequence of the first gene sequence amplification primer set, the fifth amplification priming subsequence of a second gene sequence amplification primer set, and the sixth amplification priming subsequence of the first set of shared sequence amplification primer all share a substantially identical sequence.
Embodiment B43. The method of any one of embodiments B24 to B42, wherein amplifying the predetermined paired sequences of interest comprises using the three sets of gene specific amplification primers in a molar ratio of 2:1:1 of the first set of shared sequence amplification primers:the set of first gene sequence amplification primers:the set of second gene sequence amplification primers.
Embodiment B44. The method of any one of embodiments B24 to B42, wherein amplifying the predetermined paired sequences of interest comprises using the three sets of gene specific amplification primers in a ratio of 2:1:1 of a weight of the first set of shared sequence amplification primers:a weight of the set of first gene sequence amplification primers:a weight of the set of second gene sequence amplification primers.
Embodiment B45. The method of any one of embodiments B1 to B44, wherein the method is performed using no more than six sets of primers.
Embodiment B46. The method of any one of embodiments B24 to B45, wherein amplifying the predetermined paired sequences of interest comprises amplifying the first gene sequences, the second gene sequences, and a third gene sequence using the single first gene sequence amplification primer, the single second gene sequence amplification primer, and the single shared sequence amplification primer.
Embodiment B47. The method of any one of embodiments B1 to B46, wherein placing the single cell into the cell lysis solution comprises exporting the single cell from a microfluidic chip.
Embodiment B48. The method of any one of embodiments B1 to B46, wherein placing the single cell into the cell lysis solution comprises depositing the single cell into the lysis solution using FACS.
Embodiment B49. The method of any one of embodiments B1 to B48, further comprising, before placing the cell in the lysis solution, selecting the cell using any suitable method commonly known in the art.
Embodiment B50. The method of embodiment B49, wherein selecting the cell based on cell-surface expression of a marker previously determined.
Embodiment B51. The method of embodiment B49 or B50, wherein selecting the cell comprises performing a positive selection.
Embodiment B52. The method of embodiment B49 or B50, wherein selecting the cell comprises performing a negative selection.
Embodiment B53. The method of any one of embodiments B1 to B52, further comprising purifying the RNA from the cell lysate.
Embodiment B54. The method of embodiment B53, wherein the RNA is purified using a bead-based purification method.
Embodiment B55. The method of embodiment B54, wherein generating first strand cDNA is performed in the presence of beads used in the bead-based purification method.
Embodiment B56. The method of embodiment B55, wherein amplifying the first strand cDNA is performed in the presence of beads used in the bead-based purification method.
Embodiment B57. The method of any one of embodiments B1 to B56, further comprising purifying the amplified first strand cDNA.
Embodiment B58. The method of any one of embodiments B1 to B57, further comprising further amplifying the alpha and beta TCR sequences to add index sequences for sequencing.
Embodiment B59. The method of any one of embodiments B1 to B58, further comprising performing the method on a plurality of single cells.
Embodiment B60. The method of embodiment B59, wherein predetermined paired sequences of interest are obtained from at least 50% of the plurality of single cells.
Embodiment B61. A kit for use in the methods according to any one of embodiments B1 to B60.
Embodiment B62. A kit comprising:
   a first RT primer set comprising one or more first RT primers;
   a second RT primer set comprising one or more second RT primers; and
   a third RT primer set comprising one or more third RT primers.
Embodiment B63. The kit of embodiment B62, further comprising:
   a first set of shared sequence amplification primers;
   a set of first gene sequence amplification primers; and
   a set of second gene sequence amplification primers.
Embodiment B64. The kit of embodiment B63, wherein the set of second gene sequence amplification primers has a single forward amplification primer and a single reverse amplification primer.
Embodiment B65. The kit of embodiment B63 or B64, further comprising one or more of: dNTP Mix, RT Buffer, RT Enzyme, RT Additive, RNase inhibitor, PCR enzyme K, in any combination thereof.

Other and further aspects and features of the disclosed embodiments will become apparent from the ensuing detailed description in view of the accompanying drawings.

### BRIEF DESCRIPTION OF THE DRAWINGS

The drawings illustrate the design and utility of embodiments of the disclosed inventions, in which similar elements are referred to by common reference numerals. These drawings are not necessarily drawn to scale. In order to better appreciate how the above-recited and other advantages and objects are obtained, a more particular description of the embodiments will be rendered, which are illustrated in the accompanying drawings. These drawings depict only typical embodiments of the disclosed inventions and are not therefore to be considered limiting of its scope.

FIG. 1 illustrates generating first strand cDNA in accordance with one embodiment of the present invention (SEQ ID NOs: 1, 2, and 88).

FIG. 2 illustrates amplifying first strand cDNA in accordance with an embodiment of the present invention (SEQ ID NOs: 1, 3, 89, and 90).

FIG. 3 illustrates amplifying and indexing TCR cDNA in accordance with an embodiment of the present invention (SEQ ID NOs: 4, 5, 6, 90, 91, and 92).

FIG. 4 illustrates generating and amplifying first strand cDNA and amplifying TCR cDNA in accordance with one embodiment of the present disclosure. 100—Target RNA; 101—poly-A tail at 3′ end; 200—first strand cDNA; 300—amplified cDNA; 10—first RT primer set; 11—first amplification priming subsequence; 12—poly-T subsequence with VN at 3′ end; 20—second RT primer set (TSO); 21 TSO/second amplification priming subsequence; 30—third RT primer set; 31-third amplification priming sequence; 40: TCR amplification primer sets; 41: first set of TCR amplification primer; 42: TCR alpha chain amplification primer; 43: TCR beta chain amplification primer.

FIG. 5 shows TCR pair recovery from the methods described herein. Total recovery (%) is constrained to the pool of attempted unloads whose T cell was successfully unpenned. Total recovery (%) is {the fraction of this pool of attempted unloads that contain a successfully sequenced TCR pair (alpha and beta TCR chain)}×100 and reported for each test run on Instrument 1 (left) and Instrument 2 (right). The data are sub-divided into unloads from T2 stimulated pens and antigen presenting beads (APB) stimulated pens. Applying a 2-tailed, paired T-test, there were no significant differences between Instrument 1 and Instrument 2 (P-value=0.48), or between T2-stimulated or bead-stimulated unloads (P-value=0.14).

FIG. 6 shows the sequences of TCR alpha chain (SEQ ID NO: 7) and the alignment with the exemplary TCR alpha chain amplification primer (SEQ ID NO: 5) according to the present disclosure. For the primer alignment, the reverse-compliment of the primer is used and non-aligning segments of the primer are not shown.

FIG. 7 shows the sequences of TCR beta1 and beta2 chains (SEQ ID NOs: 8 and 9) and the alignment with the exemplary TCR beta chain amplification primer (SEQ ID NO: 6) according to the present disclosure. For the primer alignment, the reverse-compliment of the primer is used and non-aligning segments of the primer are not shown.

### DETAILED DESCRIPTION

This specification describes exemplary embodiments and applications of the disclosure. The disclosure, however, is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion. In addition, as the terms “on,” “attached to,” “connected to,” “coupled to,” or similar words are used herein, one element (e.g., a material, a layer, a substrate, etc.) can be “on,” “attached to,” “connected to,” or “coupled to” another element regardless of whether the one element is directly on, attached to, connected to, or coupled to the other element or there are one or more intervening elements between the one element and the other element. Also, unless the context dictates otherwise, directions (e.g., above, below, top, bottom, side, up, down, under, over, upper, lower, horizontal, vertical, “x,” “y,” “z,” etc.), if provided, are relative and provided solely by way of example and for ease of illustration and discussion and not by way of limitation. In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent. In the context of polynucleotide sequences, “substantially identical” means that there is at least 80% agreement between a first sequence and a second sequence. Likewise, “substantially complementary” means that there is at least 80% identity between a first sequence and the complementary sequence of a second sequence. In certain embodiments, substantially identical sequences can share at least 85%, at least 90%, at least 95%, or even 100% sequence identity. Differences in sequences (or subsequences thereof) can reflect insertions, deletions, and/or substitutions at one or more nucleotide positions in one or both of the sequences being compared.

### I. DEFINITIONS

The term “ones” means more than one. As used herein, the term “plurality” can be 2, 3, 4, 5, 6, 7, 8, 9, 10, or more.

As used herein: μm means micrometer, μm³ means cubic micrometer, pL means picoliter, nL means nanoliter, and μL (or uL) means microliter.

As used herein, the term “disposed” encompasses within its meaning “located.”

A colony of biological cells is “clonal” if all of the living cells in the colony that are capable of reproducing are daughter cells derived from a single parent cell. In certain embodiments, all the daughter cells in a clonal colony are derived from the single parent cell by no more than 10 divisions. In other embodiments, all the daughter cells in a clonal colony are derived from the single parent cell by no more than 14 divisions. In other embodiments, all the daughter cells in a clonal colony are derived from the single parent cell by no more than 17 divisions. In other embodiments, all the daughter cells in a clonal colony are derived from the single parent cell by no more than 20 divisions. The term “clonal cells” refers to cells of the same clonal colony.

As used herein, a “colony” of biological cells refers to 2 or more cells (e.g., about 2 to about 20, about 4 to about 40, about 6 to about 60, about 8 to about 80, about 10 to about 100, about 20 to about 200, about 40 to about 400, about 60 to about 600, about 80 to about 800, about 100 to about 1000, or greater than 1000 cells).

As used herein, the terms “maintaining a cell” and “maintaining cells” refer to providing an environment comprising both fluidic and gaseous components and, optionally a surface, that provides the conditions necessary to keep the cell(s) viable and/or expanding. As used herein, the term “expanding” when referring to cells, refers to increasing in cell number.

A “component” of a fluidic medium is any chemical or biochemical molecule present in the medium, including solvent molecules, ions, small molecules, antibiotics, nucleotides and nucleosides, nucleic acids, amino acids, peptides, proteins, sugars, carbohydrates, lipids, fatty acids, cholesterol, metabolites, or the like.

As used herein, “T cell receptor” of “TCR” refers to a protein complex found on the surface of T lymphocytes (or “T cells”) that recognizes antigen peptides presented by major histocompatibility complex (MHC) molecules. The binding between TCR and antigen peptides is of relatively low affinity. Moreover, many TCRs recognize the same antigen peptide and many antigen peptides are recognized by the same TCR. TCRs are heterodimers that comprise two different polypeptide chains. In humans, 95% of T cells express TCRs consisting of an alpha (α) chain and a beta (β) chain (encoded by the TRA and TRB genes, respectively); the other 5% of T cells express TCRs consisting of gamma and delta (γ/δ) chains (encoded by the TRG and TRD genes, respectively). Orthologues of the TRA, TRB, TRG, and TRD loci have been mapped in various species. Alpha chain (or α chain) contains variable (V), joining (J) and “constant” C segments. Beta chain (or β chain) contains V, D, J, and diversity (D) segment. TCR clonotype is a unique nucleotide sequence that arises during the gene rearrangement process for that receptor. The combination of nucleotide sequences for the surface expressed receptor pair would define the T cell clonotype. Each locus can produce a variety of polypeptides with constant and variable regions. When the TCR engages with MHC-bound antigenic peptide, the T lymphocyte is activated through signal transduction.

An antigen, as referred to herein, is a molecule or portion thereof that can bind with specificity to another molecule, such as an Ag-specific receptor. Antigens may be capable of inducing an immune response within an organism, such as a mammal (e.g., a human, mouse, rat, rabbit, etc.), although the antigen may be insufficient to induce such an immune response by itself. An antigen may be any portion of a molecule, such as a conformational epitope or a linear molecular fragment, and often can be recognized by highly variable antigen receptors (B-cell receptor or T-cell receptor) of the adaptive immune system. An antigen may include a peptide, polysaccharide, or lipid. An antigen may be characterized by its ability to bind to a TCR or an antibody's variable Fab region. Different TCRs and antibodies have the potential to discriminate among different epitopes present on the antigen surface, the structure of which may be modulated by the presence of a hapten, which may be a small molecule.

As used herein, any oligonucleotide comprise deoxyribonucleotide such as deoxyadenosine (A), deoxyguanosine (G), and thymidine (T).

As used herein, “N” used to denote a single nucleotide, is a nucleotide selected from A, C, G, and T.

As used herein, “V” used to denote a single nucleotide, is a nucleotide selected from A, G, and C, and does not include T.

As used herein, A, C, T, G followed by “*” indicates phosophorothioate substitution in the phosphate linkage of that nucleotide.

As used herein, rG denotes a ribonucleotide included within a nucleic acid otherwise containing deoxyribonucleotides. A nucleic acid containing all ribonucleotides may not include labeling to indicate that each nucleotide is a ribonucleotide, but is made clear by context.

As used herein, the terms “primer” and “oligo” are used interchangeably to refer to an oligonucleotide sequence which can bind to a nucleic acid template sequence. Primers, which are discussed further below, can facilitate template-based extension of the primer by a polymerase or primer-based extension of the template sequence (as in the case of Template Switching Oligos (or “TSOs”)).

As used herein, a “priming sequence” is an oligonucleotide sequence which can be part of a larger oligonucleotide but, when separated from the larger oligonucleotide such that the priming sequence includes a free 3′ end, can function as a primer in a DNA (or RNA) polymerization reaction.

As used herein, a “homolog” is a gene that exhibits sequence similarity to another gene that reflects a common ancestral gene. Two genes can have common ancestry because of, for example, a speciation event (orthologs), a duplication event (paralogs), a mutagenesis event (e.g., caused by a mutagen or an error introduced during replication or DNA repair), a targeted gene editing event, or the like.

As used herein, a “paralog” is a gene that is related to another gene in the same organism by descent from a single ancestral gene that was duplicated and that may have a different DNA sequence and biological function.

As referred to herein, a region of a gene sequence that is highly diverse, large, or novel, is referred to as an “unknown region.” Thus, an unknown sequence can be a sequence that has never been sequenced before, or it can be a sequence that has been sequenced before but it is nevertheless unknown in the sense that it exhibits variation, either in its sequence (e.g., it may contain a region of hypervariable sequence) or with regard to another sequence to which it is juxtaposed, whether by genetic recombination, alternative splicing, or the like. When the unknown sequence is juxtaposed with another sequence which is known, the other sequence can be referred to herein as a “known region” or “known nucleic acid sequence.”

### II. METHODS OF AMPLIFICATION OF PAIRED TRANSCRIPT SEQUENCES FROM A SINGLE CELL

The disclosed methods provide for amplification of paired transcript sequences, such as paired T cell receptor (TCR) sequences, from an RNA sample obtained from a single cell source. The paired sequences can be TCR alpha and beta transcripts or TCR gamma and delta transcripts obtained from a single T cell. The following disclosure focuses on TCR sequences, and particularly TCR alpha and beta sequences, but it should be understood that persons skilled in the art would understand how to apply the disclosed methods to TCR gamma and delta sequences, sets of transcripts unrelated to TCRs, and cell types other than T cells. The disclosed methods can be extended to a wide variety of genes having multiple isoforms/splice forms, etc.

With regard to paired TCR sequences, a set of alpha primers and a set of beta primers provides for anchoring to a portion of a known end of the respective TCR alpha and beta chain genes (e.g., a portion of the constant region of the TCR alpha or beta chain). When used in conjunction with a first set of TCR amplification primers which bind to an amplification priming sequence, the complement which is introduced into the first strand cDNA by a template switching oligo (TSO) during the reaction generating first strand cDNA from the purified RNA, sequences from the TCR alpha and beta chain genes may be amplified reliably, in a single reaction. The resulting amplified paired alpha and beta TCR sequences may then be prepared for further analysis, such as sequencing. This approach provides the generalizable ability to sequence genes that undergo complex gene recombination events, such as the genes of T cell receptors.

In the disclosed methods nucleic acid including mRNA may be captured from a biological cell, such as a T cell, and nucleic acids (e.g., first strand cDNA) may be synthesized from the original template nucleic acid. In one embodiment, the nucleic acids are synthesized using a template switching oligonucleotide. The synthesized nucleic acids may be amplified, and the amplified nucleic acids, which may be cDNA, may then undergo a gene-specific amplification process using amplification primers configured to select for and amplify targeted gene sequences, for example genes coding for alpha and beta chains of T cell receptors. In such a gene-specific amplification process, primers can include a 3′ subsequence configured to select for and amplify targeted gene sequences.

The amplified targeted gene sequences resulting from the gene-specific amplification process may be fragmented or tagmented. Alternatively, the amplified nucleic acids or amplified targeted gene sequences may be converted into a different class of nucleic acid, such as RNA, which may be fragmented and reverse transcribed to provide fragmented DNA molecules. In either scenario, a plurality of differentially truncated nucleic acids can be produced. The plurality of differentially truncated nucleic acids may be further modified, such as by amplification and insertion of sequencing adapters, priming sequences, index molecules and/or barcodes to provide a DNA library suitably sized and adapted for parallel sequencing. Generally, a nucleic acid library suitable for sequencing may be prepared using various methods, for example those described in WO 2019/191459, published Oct. 3, 2019, the entire contents of which are incorporated herein by reference. The approaches shown here may be adapted for eventual use with various next generation sequencing platforms, such as Illumina® sequencing by synthesis chemistries, but the methods are not so limited. Any sort of sequencing chemistries may be suitable for use within these methods and may include emulsion PCR, sequencing by synthesis, pyrosequencing and semiconductor detection. One of skill in the art can adapt the methods described herein to obtain amplified nucleic acids for use with other massively parallel sequencing platforms and chemistries, such as PacBio long read systems (SMRT, Pacific Biosystems), Ion Torrent (ThermoFisher Scientific), Roche 454, Oxford Nanopore, and the like.

As mentioned above, the methods described herein may be suitably adapted for use to amplify paired sequences other than those related to alpha and beta TCR sequences and may be used on cell types other than T cells. Some suitable genes of interest may include integrins (e.g., paired alpha/beta integrin sequences), protocadherins, antibodies, DSCAMs, or anything that has variability at the 5′ end. Paired sequences may be any pair of sequences that form a complex together, e.g. a receptor complex. They may be structurally related or may be functionally related, such as genes in a signaling pathway. Alternatively, the paired sequences may be sequences of interest to an investigator and may be neither structurally nor functionally related but may represent paired sequences of interest because they are co-expressed in a single cell. Paired transcript sequences may include homologs, paralogs or variants of a gene sequence. Variant gene sequences can be alleles of a single gene, mutant sequences (e.g., arising as a result of a mutagen, an error introduced during DNA replication or DNA repair), gene edited sequences, or the like.

In some embodiments, the paired transcript sequences may have a portion of sequence that is substantially the same as the other members of the pair, while containing a second portion of sequence that is substantially different. In some embodiments, there may be three or more variants that may be amplified by these methods. These methods are not limited being performed on T cells but may be performed on any cell of interest wherein examination of multiple isoforms/splice variants may be desired.

Accordingly, a method is provided for amplification of predetermined paired sequences of interest from a single cell, including: placing a single cell into a cell lysis solution to provide a cell lysate comprising RNA; generating first strand cDNA from the RNA; amplifying the first strand cDNA to provide amplified cDNA; and amplifying the predetermined paired sequences of interest from the amplified cDNA in a single reaction, wherein the paired sequences are amplified using three sets of gene specific amplification primers. In some embodiments, amplifying the predetermined paired sequences of interest comprises amplifying two or more homologous sequences, two or more paralogous sequences, or two or more variants. The cells may be previously selected, either positively or negatively, using any suitable method, some of which are described herein. The cells may be primary cells, derived from cell lines, and/or modified, e.g., gene edited or otherwise modified. The cells may be sourced from FACs, or microfluidic platforms enabling handling of single cells.

In some embodiments, the paired sequences are amplified using three sets of gene-specific amplification primers. More generally, for n paired (or grouped) sequences, n+1 or fewer (e.g., n) gene specific amplification primer sets can be used to amplify the paired sequences. With the relationship being that for n (e.g., 3, 4, 5, etc.) paired (or grouped) sequences, the number of gene specific amplification primer sets needed to amplify the paired (or grouped) sequences is n+1 or fewer, where fewer than n+1 gene specific amplification primer sets may be sufficient when two or more of the paired (or grouped) sequences are homologs of one another that share a conserved “constant” region. For example, if the number of grouped sequences is three and two of the three are homologs with a conserved constant region, then only two gene specific amplification primer sets may be sufficient to perform the method disclosed herein. Likewise, if the number of grouped sequences is four and three of the four are homologs of one another with a conserved constant region (or, alternatively, there may be two groups of two homologs with each pair of homologs having a conserved constant region), then only three gene specific amplification primer sets may be sufficient to perform the method. Persons skilled in the art will readily understand how to extend this method to grouped sequences of five or more.

In some embodiments, placing the single cell into the cell lysis solution comprises exporting the single cell from a microfluidic chip, as described at least in U.S. Pat. No. 9,617,145 B2, entitled “Exporting A Selected Group of Micro-Objects From a Micro-Fluidic Device”, filed Oct. 14, 2014, the disclosure of which is incorporated herein by reference in its entirety. In some embodiments, placing the single cell into the cell lysis solution comprises depositing the single cell into the lysis solution using FACS.

In some embodiments, generating first strand cDNA may include: performing a reverse transcription (RT) reaction by contacting the RNA with a first RT primer set, a second RT primer set, and an enzyme having reverse transcriptase activity, for a first period of time and under conditions that generate first strand cDNA. In some embodiments, the RNA may be contacted with the first RT primer set and the enzyme having reverse transcriptase activity prior to being contacted with the second RT primer set.

In some embodiment, the RNA may be maintained at a temperature of about 10° C. or prior to being contacted with the enzyme having reverse transcriptase activity. In some embodiment, the RNA is maintained at a temperature of about 10° C., about 9° C., about 8° C., about 7° C., about 6° C., about 5° C., about 4° C., or less prior to being contacted with the enzyme having reverse transcriptase activity.

The first RT primer set may include one or more first RT primers, each first RT primer comprising a poly-T subsequence. The poly-T subsequence of each first RT primer of the first RT primer set may include a string of at least 20 thymidine nucleotides (e.g., deoxyribonucleotides). Each first RT primer of the first RT primer set may include a 3′ terminal VN subsequence, wherein V is any nucleotide selected from A, C, and G, wherein N is any nucleotide selected from A, C, G, and T, and wherein the poly-T subsequence is located 5′ to the 3′ terminal VN subsequence. each first RT primer of the first RT primer set may further include a first amplification priming subsequence. The first amplification priming subsequence may be located 5′ to a/the poly-T subsequence.

The second RT primer set may include one or more template switching oligos (TSOs). Each TSO of the second RT primer set may include a 3′ terminal ribonucleotide subsequence. The 3′ terminal ribonucleotide subsequence may include a string of at least three ribonucleotides. In some embodiments, the 3′ terminal ribonucleotide subsequence may include a rGrGrG sequence. Each TSO of the second RT primer set may further include a second amplification priming subsequence located 5′ to the 3′ terminal ribonucleotide subsequence. The first amplification priming subsequence of the first RT primer set and the second amplification priming subsequence of the second RT primer set may have substantially identical nucleotide sequences. In some embodiments, each TSO of the second RT primer set may include a barcode subsequence, which may be like any barcode subsequence as described herein. When present, the barcode subsequence may be located 5′ to a/the terminal ribonucleotide subsequence and 3′ to a/the second amplification priming subsequence. In some embodiments, the second RT primer set may include a single TSO, having any combination of features as described herein.

Amplifying the first strand cDNA may include contacting the first strand cDNA with a third primer set comprising one or more third primers, for a second period of time and under conditions that generate amplified cDNA. Each third primer of the third primer set may include a third amplification priming subsequence. In some embodiments, the first amplification priming subsequence of the first RT primer set, the second amplification priming subsequence of the second RT primer set, and the third amplification priming subsequence of the third primer set may all have substantially identical nucleotide sequences. In some embodiments, the third primer set may be a single primer.

Generating first strand cDNA and amplifying the first strand cDNA may be performed without an intervening purification of the first strand cDNA.

In some embodiments, the methods described herein further comprise purifying the RNA from the cell lysate. In some embodiments, the RNA is purified using a bead-based purification method, as described at least in PCT Publication No. WO2019191459 A1, entitled “Methods for Preparation of Nucleic Acid Sequencing Libraries”, filed on Mar. 28, 2019, which is incorporated herein by reference in its entirety. In further embodiments, generating first strand cDNA is performed in the presence of beads used in the bead-based purification method.

In some embodiments, the methods described herein further comprise purifying generated first strand cDNA. In further embodiments, the generated first strand cDNA is purified using a bead-based purification method. In further embodiments, amplifying the first strand cDNA is performed in the presence of beads used in the bead-based purification method.

In some embodiments, the methods described herein further comprise purifying the amplified first strand cDNA. In further embodiments, the amplified first strand cDNA is purified using a bead-based purification method.

In some embodiments, generating first strand cDNA and amplifying the first strand cDNA are performed without an intervening purification of the first strand cDNA.

In some embodiments, the methods disclosed herein further comprise gene-specific amplification using amplification primers configured to select for and amplify targeted gene sequences.

Amplifying the predetermined paired sequences of interest may include contacting the amplified cDNA with the three sets of gene specific amplification primers for a third period of time and under conditions that provide amplification of the predetermined paired sequences of interest from the amplified cDNA, wherein the three sets of gene specific amplification primers may include a first set of shared (e.g., for each of the isoforms/splice forms or variants, the sequence is substantially the same) sequence amplification primers, a set of first gene sequence amplification primers, and a set of second gene sequence amplification primers.

In some embodiments, amplifying the predetermined paired sequences of interest comprises using the three sets of target-specific amplification primers in a molar ratio of 2:1:1 of the first set of shared sequence amplification primers:the set of first gene sequence amplification primers:the set of at least a second gene sequence amplification primers. In some embodiments, amplifying the target-specific sequences comprises using the three sets of target-specific amplification primers in a ratio of 2:1:1 of a weight of the first set of shared sequence amplification primers:the set of first gene sequence amplification primers:the set of second gene sequence amplification primers.

Each primer of the set of first gene sequence amplification primers may include a first gene specific complement subsequence that is substantially identical to a complement sequence of a constant region sequence of a first gene specific sequence. The constant region sequence of the first gene specific sequence may be located at or proximal to a junction with a variable region sequence of the first gene specific sequence. Each primer of the set of first gene sequence amplification primers may further include a fourth amplification priming subsequence located 5′ to the first gene specific complement subsequence. Each primer of the set of first gene sequence amplification primers may further include a barcode subsequence (e.g., a string of N nucleotides and/or a unique molecular identifier (UMI)) subsequence. In some embodiments, the set of first gene sequence amplification primers may be a single first gene sequence amplification primer.

Each primer of the set of second gene sequence amplification primers may include a second gene specific complement subsequence that is substantially identical to a complement sequence of a constant region sequence of a second gene specific sequence. The constant region sequence of the second gene specific sequence may be located at or proximal to a junction with a variable region sequence of the second gene specific sequence. Each primer of the set of second gene sequence amplification primers may further include a fifth amplification priming subsequence located 5′ to the second gene specific complement subsequence. Each primer of the set of second gene sequence amplification primers may further include a barcode subsequence and/or a unique molecular identifier (UMI) subsequence. The set of second gene sequence amplification primers may be a single second gene sequence amplification primer. In some embodiments, amplifying the predetermined paired sequences of interest comprising a first gene specific sequence, a second gene specific sequence, optionally a third gene specific sequence may include amplifying both the second gene specific sequence and the third gene specific sequence using the single second gene sequence amplification primer.

Each primer of the first set of shared sequence amplification primers may include an anchor subsequence that is substantially identical to at least a subsequence of each TSO of the second RT primer set. A 3′ terminal subsequence of each primer of the first set of shared gene sequence amplification primers may comprise a TG-3′ or a TGG-3′ sequence. Each primer of the first set of shared gene sequence amplification primers may further include a sixth amplification priming subsequence located 5′ to the anchor subsequence. Each primer of the first set of shared sequence amplification primers may further include a barcode subsequence and/or a unique molecular identifier subsequence.

In some embodiments, the fourth amplification priming subsequence of the first gene sequence amplification primer set, the fifth amplification priming subsequence of a second gene sequence amplification primer set, and the sixth amplification priming subsequence of the first set of shared sequence amplification primer may all share a substantially identical sequence.

In some embodiments, amplifying the predetermined paired sequences of interest may include using the three sets of gene specific amplification primers in a molar ratio of 2:1:1 of the first set of shared sequence amplification primers:the set of first gene sequence amplification primers:the set of second gene sequence amplification primers. In other embodiments, amplifying the predetermined paired sequences of interest may include using the three sets of gene specific amplification primers in a ratio of 2:1:1 of a weight of the first set of shared sequence amplification primers:a weight of the set of first gene sequence amplification primers:a weight of the set of second gene sequence amplification primers.

In some embodiments, the method may be performed using no more than six sets of primers.

In some embodiments, the barcode sequence disclosed herein comprises a string of N nucleotides. In some embodiments, the UMI subsequence disclosed herein comprises a string of N nucleotides. In further embodiments, N is 2 to 10, 3 to 8, 3 to 6, or 2, 3, 4, 5, 6, 7, 8, 9, or 10 nucleotides. In further embodiments, each N in the string is any nucleotide. In further embodiments, each N is selected from any deoxyribonucleotide. In further embodiments, each N is selected from A, C, G, and T.

In some embodiments, the fourth amplification priming subsequence, the fifth amplification priming subsequence, and the sixth amplification priming subsequence set all have substantially identical sequences. In some embodiments, the fourth amplification priming subsequence and the fifth amplification priming subsequence have substantially identical nucleotide sequences. In some embodiments, the fourth amplification priming subsequence and the six amplification priming subsequence have substantially identical nucleotide sequences. In some embodiments, the fifth amplification priming subsequence and the sixth amplification priming subsequence have substantially identical nucleotide sequences.

In some embodiments, the method is performed using no more than six sets of primers.

In some embodiments, the methods described herein further comprise amplifying the target transcript sequences to add index sequences (e.g., to facilitate next generation sequencing (NGS)).

In some embodiments, the methods described herein further comprise performing the method on a plurality of single cells. The plurality of single cells may be 2, 3, 4, 5, 6, 7, 8, 9, 10, 15, 20, 25, 30, 40, 48, 50, 60, 70, 80, 90, 96, or more of single cells. In some embodiments, paired target transcript sequences are obtained from at least 50%, at least 55%, at least 60%, at least 65%, at least 70%, at least 75%, at least 80%, at least 85%, at least 90% or more of the plurality of single cells.

A. Exemplary Methods

For any of the methods described herein, T cell(s), or other cells, may be exported from a microfluidic device or any other kind of cell holder to a well of a wellplate (or other receptacle), where the T cell may be present as a single T cell or as more than one T cell. In cases where there is more than one T cell, the plurality of T cells may be a clonal population of cells. The T cell may be any suitable type of T cell (e.g., any of the T cell types disclosed herein, including any of the exemplary cell types disclosed herein, but the method is not limited to the exemplary cell types described herein.

The T cell may be lysed using any suitable method and reagents to effect lysis of the cell membrane, thereby making RNA molecules available for capture. The RNA molecules may be captured to a capture object such as a bead, which may be paramagnetic or may not be paramagnetic.

In some embodiments, lysis of the T cell may be performed while the T cell is disposed within a microfluidic device, and RNA molecules (which may be any kind of RNA, including mRNA) may be captured by a capture object. In some embodiments, the T cell(s) may be disposed within an isolation region of a sequestration pen, as generally described in WO 2015/061497, published Apr. 30, 2015, WO 2015/095623, published Jun. 25, 2015, and WO 2018/064640, published May 31, 2018, the entire contents of each of which is incorporated herein by reference. The capture object bearing captured RNA molecules may be exported from the microfluidic device and processing may continue as for RNA molecules which are originally captured to a capture object in a well plate. In some embodiments, the capture object may have a barcode which may be read on chip and also read from sequencing a portion of cDNA off chip, thereby allowing sequencing data from a well of the well plate to be correlated with the sequestration pen of the microfluidic device from which the capture object was exported.

No matter the manner in which RNA molecules are provided to the well plate for the methods, the RNA molecules are reverse transcribed to provide cDNA. In some embodiments, RNA molecules may be captured/primed with a set of primers having one or more degenerate nucleotide positions. In particular, the set of primers can have a 3′ terminal dTVN oligonucleotide sequence, wherein the T is thymidine, and V denotes a C (cytidine), G (guanosine), or A (adenosine), and N represents A, G, T, or C. In some embodiments, the set of primers may further include a 5′-biotin moiety.

The reverse transcription (RT) reaction used to generate the first strand cDNA from the RNA molecules also may include a Template Switching Oligonucleotide (TSO), which, optionally may be 5′-biotinylated. The TSO or bio TSO may further include additional nucleotides to help amplify specific desired amplicons, such as TCR specific amplicons. The TSO or bio TSO may further be a nested TSO. The product of the RT reaction is a plurality of cDNA molecules, which are used in any of the methods.

For any of the methods, one or more barcodes may be introduced, permitting multiplexing of the sequencing experiments. In certain embodiments, a first barcode sequence may be unique for the mRNA molecule isolated from a T cell. In certain embodiments, the described methods may be used in preparing amplified nucleic acids which are used in preparing a nucleic acid library for sequencing. For example, these methods may also be adapted to suitably provide fragmented nucleic acids for a 5′ anchored sequencing library or a 3′ anchored sequencing library.

Due to use of the alpha chain amplification primers and the beta chain amplification primers, the amplification products may therefore have substantially only products specific to the TCR alpha and beta chains, thereby providing a gene specific nucleic acid library. In some embodiments, the gene specific nucleic acid library may be a library encoding paired TCR sequences. In some embodiments, the TCR library may include both alpha and beta chain sequences, or both gamma and delta chain sequences. In some embodiments, rather than using all three sets of TCR amplification primers in a single reaction, only the first set of TCR amplification primers and either the set of alpha chain amplification primers or the set of beta chain amplification primers is used. Thus, in some embodiments, the alpha TCR sequences are amplified using two sets of TCR amplification primers (the first set of TCR amplification primers and the set of alpha chain amplification primers). In some embodiments, the beta TCR sequences are amplified using two sets of TCR amplification primers (the first set of TCR amplification primers and the set of beta chain amplification primers). In some embodiments, the alpha and beta TCR sequences are amplified by amplifying the alpha TCR sequences and the beta TCR sequences separately and then combining the resulting solutions to give the combined amplified alpha and beta TCR sequences.

In some embodiments, the method is performed using no more than six sets of primers.

Accordingly, in some embodiments, methods for paired amplification of alpha and beta T cell receptor (TCR) sequences from a single T cell are provided. In some embodiments, the method comprises (a) placing a single T cell into a cell lysis solution to provide a T cell lysate comprising RNA; (b) generating first strand cDNA from the RNA; (c) amplifying the first strand cDNA to provide amplified cDNA; and (d) amplifying the alpha and beta TCR sequences from the amplified cDNA in a single reaction. In some embodiments, the alpha and beta TCR sequences are amplified using three sets of TCR amplification primers.

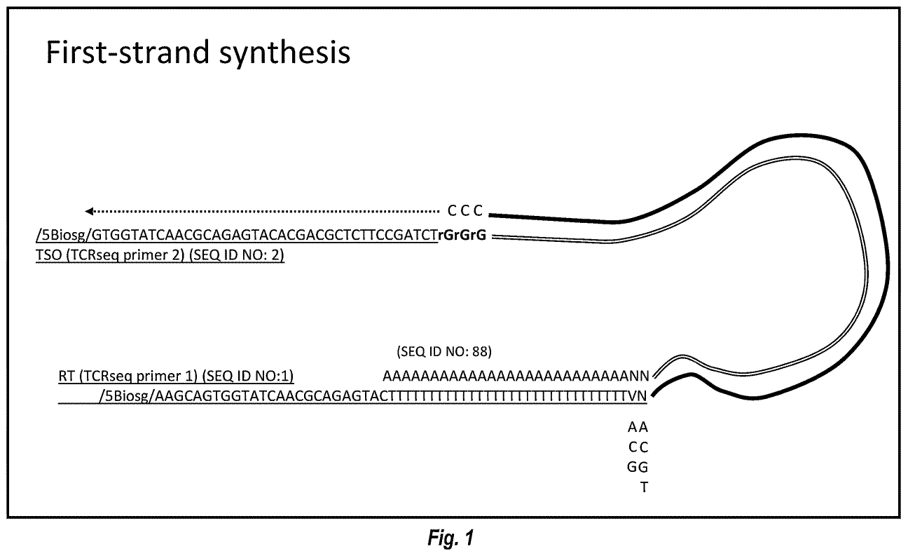

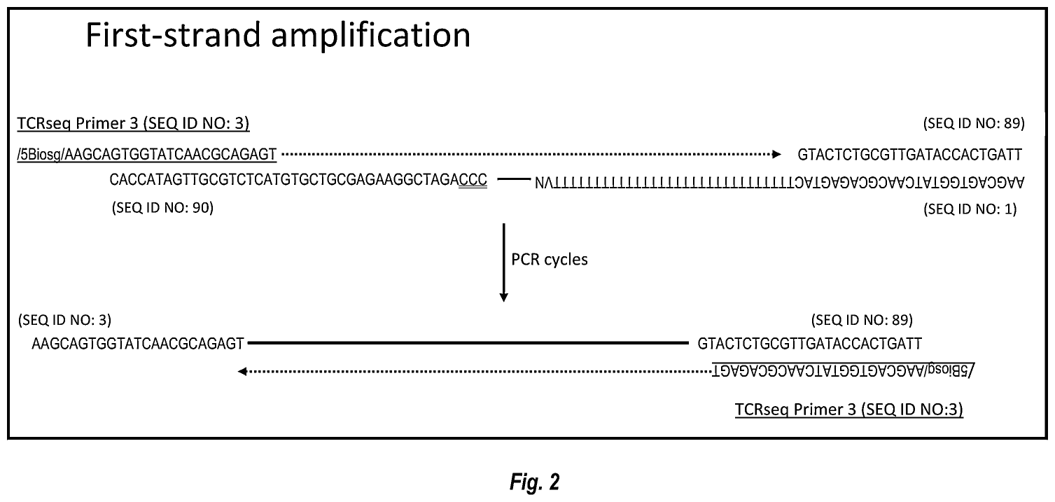

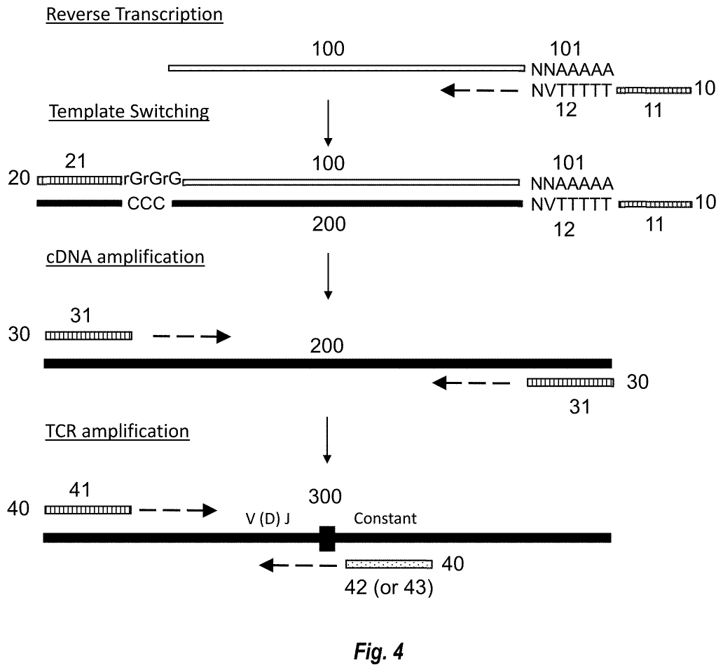

FIGS. 1-4 illustrates generating and amplifying first strand cDNA and amplifying TCR cDNA in accordance with certain embodiments of the methods described herein.

B. Single Cell Preparation

In some embodiments, placing the single T cell into the cell lysis solution comprises exporting the single T cell from a microfluidic chip. In some embodiments, placing the single T cell into the cell lysis solution comprises depositing the single T cell into the lysis solution using FACS.

In some embodiments, the methods described herein further comprise, before placing the T cell in the lysis solution, selecting the T cell based on cell-surface expression of a marker selected from CD3, CD4, CD8, CD45RO, CD45RA, CCR7, CD62L, PD-1, CD137, or any combination thereof.

In some embodiments, the methods described herein further comprise, before placing the T cell in the lysis solution, selecting the T cell based on expression by the T cell of one or more cytokines. In further embodiments, the one or more cytokines is selected from IFNgamma, TNFalpha, IL2, or a combination thereof.

In some embodiments, selecting the T cell comprises performing a positive selection. In some embodiments, selecting the T cell comprises performing a negative selection. In some embodiments, the single T cell is selected based on being positive for cytokine secretion. In some embodiments, the single T cell is selected based on being positive for surface-expressed marker secretion.

While the T cells to be sequenced may be provided from any kind of workflow and/or any kind of apparatus used to enrich, and optionally stimulate the cells, some additional advantages may be obtained when the T cells to be sequenced are enriched, cultured and/or stimulated in any combination within a microfluidic device such as, but not limited to, an OptoSelect™ chip (Berkeley Lights, Inc. Emeryville Calif.), some elements of which include but are not limited to the features of the microfluidic devices described in U.S. Pat. No. 9,857,333 B2, entitled “Pens for Biological Micro-Objects”, filed on Oct. 22, 2013; U.S. Pat. No. 10,010,882 B2, entitled “Microfluidic Devices Having Isolation Pens and Methods of Testing Biological Micro-Objects with Same”, filed on Oct. 22, 2014; U.S. Pat. No. 10,723,998 B2, entitled “Microfluidic Cell Culture”, filed on Apr. 22, 2016; and U.S. application Ser. No. 16/196,649, entitled “Covalently Modified Surfaces, Kits, and Methods of Preparation and Use”, filed on Nov. 20, 2018, each of which disclosures are herein incorporated by reference in its entirety.

In particular, prior sorting, enrichment and/or stimulation of T cells, including but not limited to U.S. application Ser. No. 15/802,100, entitled “Selection and Cloning of T Lymphocytes in a Microfluidic Device”, filed on Nov. 2, 2017; U.S. application Ser. No. 16/253,869, entitled “Sorting of T Lymphocytes in a Microfluidic Device”, filed on Jan. 22, 2019; U.S. application Ser. No. 16/987,835, entitled “Mutant IDH1 Specific T Cell Receptor” filed on Aug. 7, 2020; and U.S. application Ser. No. 16/743,849, entitled “Antigen-Presenting Synthetic Surfaces, Covalently Functionalized Surfaces, Activated T Cells and Uses Thereof”, filed on Jan. 15, 2020, each of which disclosures is herein incorporated by reference in its entirety, may be performed within the OptoSelect chip.

The OptoSelect chip includes a channel and chambers (e.g., sequestration pens) connected thereto, providing the possibility of performing assays upon the T cells within the microfluidic device prior to sequencing. The T cells, which may be disposed each within a separate chamber, may be tested using functional assays or phenotypic assays, including but not limited to: U.S. application Ser. No. 16/743,849, entitled “Antigen-Presenting Synthetic Surfaces, Covalently Functionalized Surfaces, Activated T Cells and Uses Thereof”, filed on Jan. 15, 2020; U.S. application Ser. No. 16/910,682, entitled “General Functional Assay”, filed on Jun. 24, 2020; International Application Serial No. PCT/US2019/059495, entitled “Methods for Assaying Biological Cells in a Microfluidic Device”, filed on Nov. 1, 2019; and International Application Serial No. PCT/US2019/051129, entitled “Methods for Assaying Binding Activity”, filed on Sep. 13, 2019, each of which disclosures is herein incorporated by reference in its entirety. Assays may be performed to identify characteristics such as, but not limited to, desired cell killing capabilities, antigenic presentation, activation status or tested to identify cell surface proteins produced by the T cell. Any of the assay data obtained for the individual T cell may be stored along with the identification information for the specific chamber of the OptoSelect Chip.

Advantageously, the connection between phenotype exhibited by the T cell and sequence of its TCR may be maintained by linking the assay data, and optionally, images of the T cell, with the destination well of the microwell plate where the T cell is lysed and prepared for sequencing. Barcoding, which may be performed during sequencing library preparation, permits linkage of the sequencing results with the original phenotypic/functional assays performed within the source chamber of the OptoSelect chip.

C. Generation of First Strand cDNA

In some embodiments, generating first strand cDNA comprises: performing a reverse transcription (RT) reaction by contacting the RNA with a first RT primer set, a second RT primer set, and an enzyme having reverse transcriptase activity, for a first period of time and under conditions that generate first strand cDNA. For example, the RT reaction may be performed for at least about 1 h, 1.5 h, 2.0 h, 2.5 h, 3.0 h, 3.5 h, or at least about 4 h, and at a temperature which may be about 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., or about 55° C. In some embodiments, wherein the RNA is contacted with the first RT primer set and the enzyme having reverse transcriptase activity prior to being contacted with the second RT primer set.

In some embodiment, the RNA is maintained at a temperature of about 10° C. or prior to being contacted with the enzyme having reverse transcriptase activity. In some embodiment, the RNA is maintained at a temperature of about 10° C., about 9° C., about 8° C., about 7° C., about 6° C., about 5° C., about 4° C., or less prior to being contacted with the enzyme having reverse transcriptase activity.

D. Amplification of First Strand cDNA

In some embodiments, amplifying the first strand cDNA comprises contacting the first strand cDNA with a third primer set comprising one or more third primers, for a second period of time and under conditions that generate amplified cDNA. For example, the amplification may be performed for from about 5 cycles of PCR, 7, 9, 11, 13, 15, 17, 19, 21, 23, 25, 27, 29, 31, 33, or about 35 cycles of PCR, depending on the amount of input cDNA and the desired amount of output cDNA. The annealing and extension temperatures may be adjusted depending on template source, PCR enzyme, and/or buffer used. Annealing temperatures may be selected to be between about 50° C., 53° C., 55° C., 57° C., 59° C., 61° C., 63° C., 65° C., 67° C., 69° C., about 70° C. or any temperature therebetween. Chain extension temperatures may be selected to be about 64° C., 65° C., 66° C., 67° C., 68° C., 69° C., 70° C., 71° C., 72° C., or any temperature therebetween. In some embodiments, generating first strand cDNA and amplifying the first strand cDNA are performed without an intervening purification of the first strand cDNA.

E. Amplifications of TCR Transcripts

In some embodiments, amplifying the alpha and beta TCR sequences comprises contacting the amplified cDNA with three sets of TCR amplification primers for a third period of time and under conditions that provide amplification of paired alpha and beta TCR sequences from the amplified cDNA. In some embodiments, the three sets of TCR amplification primers comprise a first set of TCR amplification primers, a set of TCR alpha chain amplification primers, and a set of TCR beta chain amplification primers, as described herein. In some embodiments, amplifying the alpha and beta TCR sequences comprises using the three sets of TCR amplification primers in a molar ratio of 2:1:1 of the first set of TCR amplification primers:the set of TCR alpha chain amplification primers:the set of TCR beta chain amplification primers. In some embodiments, amplifying the alpha and beta TCR sequences comprises using the three sets of TCR amplification primers in a ratio of 2:1:1 of a weight of the first set of TCR amplification primers:a weight of the set of TCR alpha chain amplification primers:a weight of the set of TCR beta chain amplification primers.

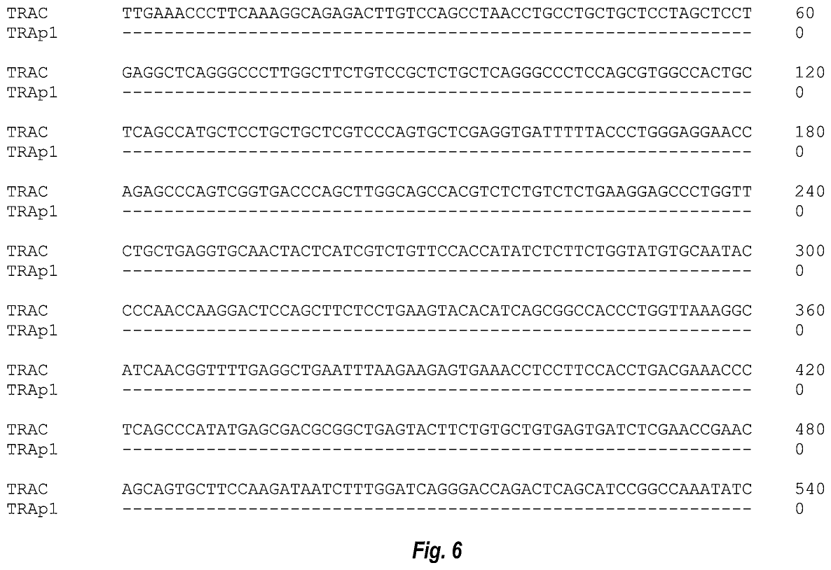

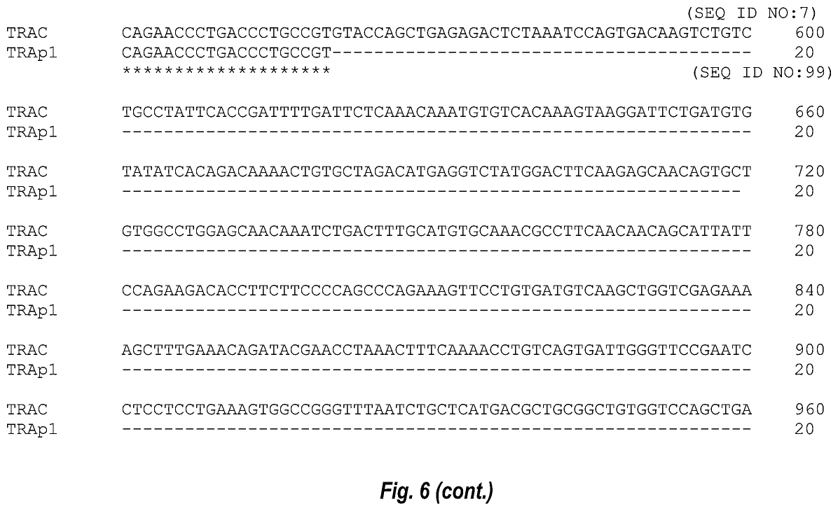

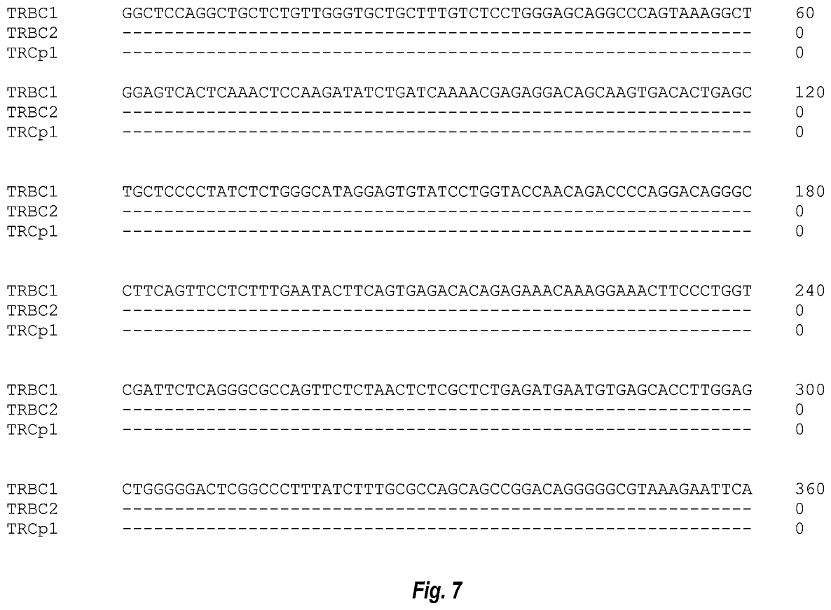

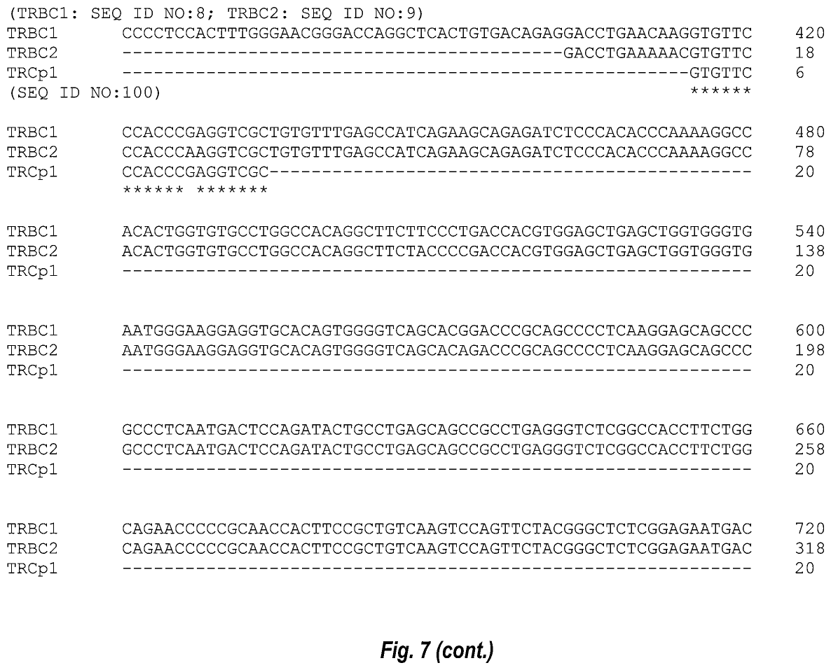

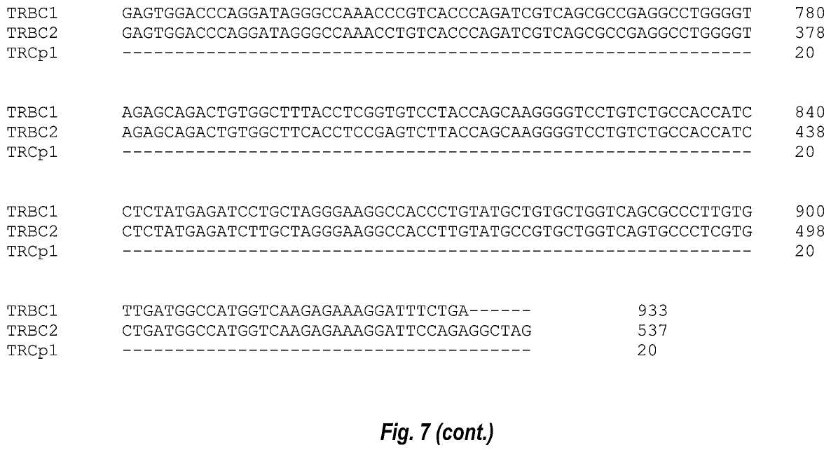

Further, Applicant has surprisingly discovered that a simplified set of primers can successfully amplify both gene forms of beta TCR sequences as well as alpha TCR sequences in the same amplification reaction. Surprisingly, beta1 and beta2 sequences can be amplified with the same 3′ primer. Thus, all of alpha, beta1 and beta2 sequences can be amplified using only three primers in total, a single 5′ primer and two 3′ primers. FIG. 6 shows the sequences of TCR alpha chain (SEQ ID NO: 7) and the alignment with the subsequence of the exemplary TCR alpha chain amplification primer (SEQ ID NO: 5) according to the present disclosure. FIG. 7 shows the sequences of TCR beta1 and beta2 chains (SEQ ID NOs: 8 and 9) and the alignment with the subsequence of the exemplary TCR beta chain amplification primer (SEQ ID NO: 6).

Accordingly, in some embodiments, the method comprises amplifying the beta chain 1 sequence and beta chain 2 sequence using the single beta chain amplification primer. In some embodiments, the method comprises amplifying the alpha TCR sequences, beta chain 1 sequence, and beta chain 2 sequence using the single TCR alpha chain amplification primer, the single beta chain amplification primer, and the single TCR amplification primer.

F. Indexing/Sequencing

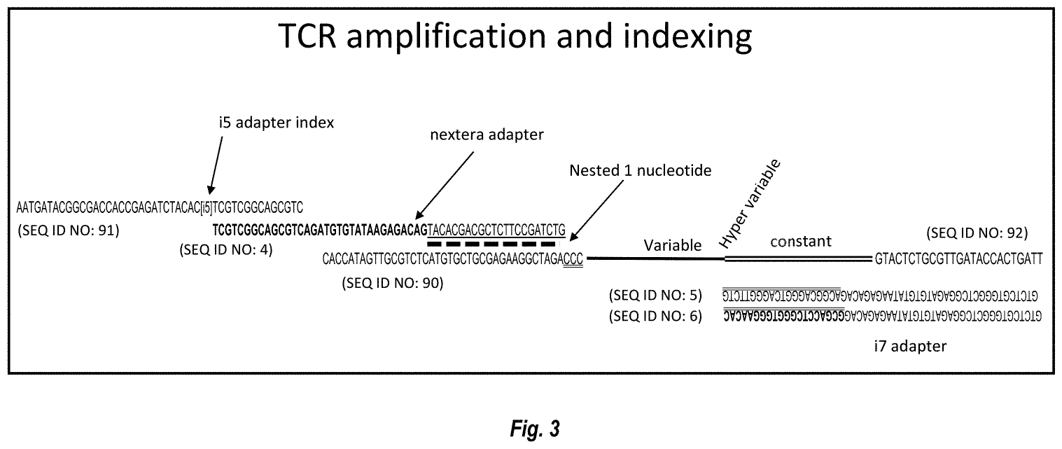

In some embodiments, the methods described herein further comprise amplifying the paired target sequences (e.g., alpha and beta TCR sequences) to add index sequences (e.g., to facilitate next generation sequencing (NGS)). An exemplary configuration of indexing that can be used for NGS is depicted in FIG. 3.

Sequencing. The methods described herein may be used for 150×150 paired-end reads on an Illumina Miseq (MiSeq Reagent Kit v2 (300 cycle) (Illumina MS-102-2002)). Data analysis first requires demultiplexing the Illumina indexes (if more than one is used), followed by sorting each BCI that identifies each sample, per index. After adapter trimming, etc. the reads may be assembled into the full length TCR (or TCR V(D)J region). The methods described herein may be used for 300×300 paired-end reads.

In some embodiments, the methods described herein further comprise performing the method on a plurality of single T cells. The plurality of single T cells may be 2, 3, 4, 5, 6, 7, 8, 9, 10, 15, 20, 25, 30, 40, 48, 50, 60, 70, 80, 90, 96, or more of single T cells. In some embodiments, paired alpha and beta T cell receptor (TCR) sequences are obtained from at least 50%, at least 55%, at least 60%, at least 65%, at least 70%, at least 75%, at least 80%, at least 85%, at least 90% or more of the plurality of single T cells.

### III. PRIMERS AND OTHER REAGENTS

A. Primer

A primer, as referred to herein, is a single stranded oligonucleotide, and may be DNA or RNA. A primer may typically be about 10 to about 50, about 12 to about 45, about 15 to about 40, about 18 to about 35, about 20 to about 30 nucleotides in length, or any number therebetween. Primers may be provided alone or in primer pairs, to prime both strands (top, bottom) of a double stranded DNA, and provide a starting point for DNA replication (e.g., strand extension). A primer may be a universal primer, a degenerate primer, or a specific primer. A “set of primers” or “prime set” as used herein, refers to a single primer or two or more primers. In some embodiments, a set of primers may comprise two or more primers are substantially identical to one another but include one or more (e.g., two, three, four, five, etc.) positions at which the primers in the set differ. Such differences can include base substitutions (such as in degenerate primers), insertions, and/or deletions. In some embodiments, the primer set is not a mixture of two or more primers having disparate sequences, e.g., not substantially identical. In some embodiments, a set of primers may be a single primer that can amplify more than one variant, homolog, or paralog of a gene or gene family.

1. First RT Primer Set

In some embodiments, the first RT primer set comprises one or more first RT primers. In some embodiments, each first RT primer comprises a poly-T subsequence. The poly-T subsequence may include a string of at least 20 thymidine nucleotides (e.g., deoxyribonucleotides). In some embodiments, the poly-T subsequence comprises a string of at least 21, at least 22, at least 23, at least 24, at least 25, at least 26, at least 27, at least 28, at least 29, at least 30, or more thymidine nucleotides. In some embodiments, the poly-T subsequence comprises a string of at least 26 thymidine nucleotides. In some embodiments, the poly-T subsequence comprises a string of at least 27 thymidine nucleotides. In some embodiments, the poly-T subsequence comprises a string of at least 28 thymidine nucleotides. In some embodiments, the poly-T subsequence comprises a string of at least 29 thymidine nucleotides. In some embodiments, the poly-T subsequence comprises a string of at least 30 thymidine nucleotides.

In some embodiments, each first RT primer of the first RT primer set comprises a 3′ terminal VN subsequence. In some embodiments, each V is a deoxyribonucleotide selected from A, C, and G. In some embodiments, each N is a deoxyribonucleotide selected from A, C, G, and T. In further embodiments, the poly-T subsequence is located 5′ to the 3′ terminal VN subsequence.

In some embodiments, each first RT primer of the first RT primer set comprises a first amplification priming subsequence. In further embodiments, the first amplification priming subsequence is located 5′ to a/the poly-T subsequence.

In some embodiments, each first RT primer of the first RT primer set comprises a first amplification priming subsequence, a poly-T subsequence, a VN subsequence.

2. Second RT Primer Set

In some embodiments, the second RT primer set comprises one or more template switching oligonucleotide.

a) Template switching oligonucleotide.

A “template switching oligonucleotide” (TSO) as used herein, refers to an oligonucleotide that permits the terminal transferase activity of an appropriate reverse transcriptase, such as, but not limited to Moloney murine leukemia virus (MMLV), to use the deoxycytidine nucleotides (e.g., CCC) added to anchor a template switching oligonucleotide. Upon base pairing between the template switching oligonucleotide and the appended deoxycytidines, the reverse transcriptase “switches” template strands from the captured RNA to the template switching oligonucleotide and continues replication to the 5′ end of the template switching oligonucleotide. Thus, a complete 5′ end of the transcribed RNA is included and additional priming sequences for further amplification may be introduced. The TSO may further include biotin linked to its 5′ end of the oligonucleotide. Exemplary TSO of the second RT primer set is listed in Table 1 (SEQ ID NO: 2). Any selected oligonucleotide sequence can be used as may be determined by one of skill in the art. Any change made to that TSO sequence may be reflected in the third primer of the third RT primer set for amplification of the first strand cDNA.

In some embodiments, the second RT primer set comprises a single TSO.

In some embodiments, each TSO of the second RT primer set comprises a 3′ terminal ribonucleotide subsequence. In further embodiments, the 3′ terminal ribonucleotide subsequence comprises a string of at least three ribonucleotides. In further embodiments, the 3′ terminal ribonucleotide subsequence comprises a rGrGrG sequence. In further embodiments, the 3′ terminal ribonucleotide subsequence comprises a rGrG sequence or a rGrGrGrG sequence.

In some embodiments, each TSO of the second RT primer set comprises a second amplification priming subsequence located 5′ to the 3′ terminal ribonucleotide subsequence. In some embodiments, the first amplification priming subsequence of the first RT primer set and the second amplification priming subsequence of the second RT primer set have substantially identical nucleotide sequences. In some embodiments, the first amplification priming subsequence of the first RT primer set and the second amplification priming subsequence of the second RT primer set have substantially different nucleotide sequences.

In some embodiments, each TSO of the second RT primer set comprises a barcode subsequence. In further embodiments, the barcode sequence comprises a string of N nucleotides. In further embodiments, N is 2 to 10, 3 to 8, 3 to 6, or 2, 3, 4, 5, 6, 7, 8, 9, or 10 nucleotides. In further embodiments, each N in the string is any nucleotide. In further embodiments, each N is selected from any deoxyribonucleotide. In further embodiments, each N is selected from A, C, G, and T.

In further embodiments, the barcode subsequence is located 5′ to a/the terminal ribonucleotide subsequence and 3′ to a/the second amplification priming subsequence.

3. Third RT Primer Set

In some embodiments, the first strand cDNA is amplified by contacting the first strand cDNA with a third primer set comprising one or more third primers, for a period of time and under conditions that generate amplified cDNA. In some embodiments, the third primer set comprises a single primer. In some embodiments, the third primer set comprises more than one primers. In some embodiments, each third primer of the third primer set comprises a third amplification priming subsequence.

In some embodiments, the first amplification priming subsequence of the first RT primer set, the second amplification priming subsequence of the second RT primer set, and the third amplification priming subsequence of the third primer set all have substantially identical nucleotide sequences. Exemplary sets of first, second, and third RT primer sets are provided in Table 1 (SEQ ID NOs: 1-3), each primer sequence of each of the first, second, and third RT primer sets having a structure as described herein. In some embodiments, the first amplification priming subsequence of the first RT primer set and the second amplification priming subsequence of the second RT primer set have substantially identical nucleotide sequences. In some embodiments, the first amplification priming subsequence of the first RT primer set and the third amplification priming subsequence of the third primer set have substantially identical nucleotide sequences. In some embodiments, the second amplification priming subsequence of the second RT primer set and the third amplification priming subsequence of the third primer set have substantially identical nucleotide sequences.

In some embodiments, the first strand cDNA is amplified by contacting the first strand cDNA with a third primer set comprising one or more third primers, for a period of time and under conditions that generate amplified cDNA. In some embodiments, the third primer set comprises a single primer. In some embodiments, the third primer set comprises more than one primers. In some embodiments, each third primer of the third primer set comprises a third amplification priming subsequence.

4. TCR Specific Amplification Primers

a) TCR Alpha Chain Amplification Primers

A TCR alpha chain amplification primer may comprise a TCR alpha chain complement subsequence that includes identity on both sides of the junction, or just on the constant region side of the junction. Accordingly, in some embodiments, each primer of the set of TCR alpha chain amplification primers comprises a TCR alpha chain complement subsequence that is substantially identical to a complement sequence of a constant region sequence of a TCR alpha chain. In some embodiments, the constant region sequence of the TCR alpha chain is located at or proximal to a junction with a variable region sequence of the TCR alpha chain. In other embodiments, a TCR alpha chain amplification primer may comprise a sequence that is substantially identical to a complement sequence of the constant region sequence and the variable region sequence proximal to the junction of the constant region sequence.

In some embodiments, each primer of the set of TCR alpha chain amplification primers further comprises a fourth amplification priming subsequence located 5′ to the TCR alpha chain complement subsequence.

In some embodiments, each primer of the set of TCR alpha chain amplification primers further comprises a barcode subsequence and/or a unique molecular identifier (UMI) subsequence. In further embodiments, the barcode sequence comprises a string of N nucleotides. In further embodiments, the UMI subsequence comprises a string of N nucleotides. In further embodiments, N is 2 to 10, 3 to 8, or 3 to 6 nucleotides, or 2, 3, 4, 5, 6, 7, 8, 9, or 10 nucleotides. In further embodiments, each N in the string is any nucleotide. In further embodiments, each N is selected from any deoxyribonucleotide. In further embodiments, each N is selected from A, C, G, and T.

In some embodiments, the set of TCR alpha chain amplification primers comprises a single TCR alpha chain amplification primer.

b) TCR Beta Chain Amplification Primers

In some embodiments, each primer of the set of TCR beta chain amplification primers comprises a TCR beta chain complement subsequence that is substantially identical to a complement sequence of a constant region sequence of a TCR beta chain.

In some embodiments, the constant region sequence of the TCR beta chain is located at or proximal to a junction with a variable region sequence of the TCR beta chain.

In some embodiments, each primer of the set of TCR beta chain amplification primers further comprises a fifth amplification priming subsequence located 5′ to the TCR beta chain complement subsequence.

In some embodiments, each primer of the set of TCR beta chain amplification primers further comprises a barcode subsequence and/or a unique molecular identifier (UMI) subsequence. In further embodiments, the barcode sequence comprises a string of N nucleotides. In further embodiments, the UMI subsequence comprises a string of N nucleotides. In further embodiments, N is 2 to 10, 3 to 8, or 3 to 6 nucleotides, or 2, 3, 4, 5, 6, 7, 8, 9, or 10 nucleotides. In further embodiments, each N in the string is any nucleotide. In further embodiments, each N is selected from any deoxyribonucleotide. In further embodiments, each N is selected from A, C, G, and T.

In some embodiments, the set of TCR beta chain amplification primers comprises a single TCR beta chain amplification primer.

c) First Set of TCR Amplification Primers

In some embodiments, each primer of the first set of TCR amplification primers comprises an anchor subsequence that is substantially identical to at least a subsequence of each TSO of the second RT primer set.

In some embodiments, a 3′ terminal subsequence of each primer of the first set of TCR amplification primers comprises a TG-3′ or a TGG-3′ sequence.

In some embodiments, each primer of the first set of TCR amplification primers further comprises a sixth amplification priming subsequence located 5′ to the anchor subsequence.

In some embodiments, each primer of the first set of TCR amplification primers further comprises a barcode subsequence and/or a unique molecular identifier (UMI) subsequence. In further embodiments, the barcode sequence comprises a string of N nucleotides. In further embodiments, the UMI subsequence comprises a string of N nucleotides. In further embodiments, N is 2 to 10, 3 to 8, 3 to 6, or 2, 3, 4, 5, 6, 7, 8, 9, or 10 nucleotides. In further embodiments, each N in the string is any nucleotide. In further embodiments, each N is selected from any deoxyribonucleotide. In further embodiments, each N is selected from A, C, G, and T.

In some embodiments, the first set of TCR amplification primers comprises a single TCR amplification primer. In other embodiments, the first set of TCR amplification primers comprises two or more TCR amplification primers.

In some embodiments, the fourth amplification priming subsequence of the TCR alpha chain amplification primer set, the fifth amplification priming subsequence of the TCR beta chain amplification primer set, and the sixth amplification priming subsequence of the first TCR amplification primer set all have substantially identical sequences.

Exemplary sets of the fourth, fifth, and six amplification priming subsequences sets are provided in Table 1 (part of SEQ ID NOs: 4-6), each priming subsequence having a structure as described herein. In some embodiments, the fourth amplification priming subsequence and the fifth amplification priming subsequence have substantially identical nucleotide sequences. In some embodiments, the fourth amplification priming subsequence and the six amplification priming subsequence have substantially identical nucleotide sequences. In some embodiments, the fifth amplification priming subsequence and the sixth amplification priming subsequence have substantially identical nucleotide sequences.

5. Modifications

The primers or any other oligonucleotides described herein may include a nucleotide including a modification. Nucleotide modifications may afford a wide range of tuneable functionality for the primers. A modification of the oligonucleotide may include non-natural nucleotide moieties or other small organic molecular moieties which provide for stable connection to a capture object as known in the art. Exemplary modifications include but are not limited to an amine-modified oligonucleotide; thiol-modified oligonucleotide, disulfide-modified oligonucleotide, hydrazide-modified succinate-modified oligonucleotide, or proprietary linker-modified oligonucleotide (commercially available or otherwise) which may be present at the 5′ or 3′ terminus of the first and/or second oligonucleotides, depending on the selected usage.

Alternatively, the primer or any other oligonucleotides described herein may include a biotin, streptavidin, or other biomolecule capable of binding to a respective binding molecule on a surface (e.g., a bead).

Further the primers or any other oligonucleotides may include an azidyl-modification or alkynyl-modification, permitting Click coupling to a reaction pair moiety on the capture object. Other modifications may include other non-nucleotide containing moieties, proximal to such terminal modifications to reduce steric interference for priming sequences, capturing sequences, barcoding sequences, labelling sequences, or any other sequence module of the primers or any other oligonucleotides.

The primers or any other oligonucleotides may include, within the respective nucleotide sequences, one or more modified nucleotide moieties which may improve the stability of the oligonucleotide to conditions used throughout the methods as described herein. The modifications may increase stability of the oligonucleotide with respect to one or more of melting temperature, affinity for a target nucleotide, resistance to a nuclease, and the like. In some alternative embodiments, modified oligonucleotides may provide for enhanced susceptibility to one or more nucleases or selective chemical, photochemical and/or thermal cleavages along its length.

The primer or any other oligonucleotides described herein can have various nucleic acid residues, such as for example, an unmodified nucleotide moiety, a modified nucleotide moiety, or any other feature as long as the polymerizing agent is capable of functioning on the primer as a viable substrate.

The primer or any other oligonucleotides described herein may include one or more modified nucleotides capable of incorporation into a primer in the place of a ribosyl or deoxyribosyl moiety. The modified nucleotides may be modified at the 2′ position of sugar moiety of the nucleoside, which may include substituted, unsubstituted, saturated, unsaturated, aromatic or non-aromatic moieties. Suitable moieties at the 2′ position include, but are not limited to, alkoxy (such as methoxy, ethoxy, propoxy), 2′-oxy-3-deoxy, 2′-t-butyldimethylsilyloxy, furanyl, propyl, pyranosyl, pyrene, acyclic moieties, and the like. In other embodiments, a 2′ modification may include a 2′ fluoro-modified nucleotide, a 2′ alkoxyalkyl (e.g., 2′Omethoxyethyl (MOE)), or the like. Further the modified nucleotide may be a locked nucleic acid (LNA), an unlocked nucleic acid or an unnatural nucleotide analog such as, but not limited to, 5-nitroindole, 5-methyl dC, Super T® (IDT), Super G® (IDT) and the like.

(1) Optional oligonucleotide sequences.

As noted above, the primers or any other oligonucleotide sequences described herein may optionally have one or more additional sequences, which either provide a landing site for primer extension or a site for immobilization to complementary hybridizing anchor sites within a massively parallel sequencing array or flow cell.

Each primer may optionally further include a unique molecule identifier (UMI) sequence. A first set of primers may have a different UMI from a second set of primers, permitting identification of unique captures as opposed to numbers of amplified sequences. In some embodiments, the UMI may be located 3′ to the priming sequence. The UMI sequence may be an oligonucleotide having about 5 to about 20 nucleotides. In some embodiments, the oligonucleotide sequence of the UMI sequence may have about 10 nucleotides.

DNA molecules produced as described herein for use in sequencing may also include additional indicia such as a pool Index sequence. The Index sequence is a sequence of 4 to 10 oligonucleotides which uniquely identify a set of nucleic acids as belonging to one experiment, permitting multiplex sequencing combining sequencing libraries from several different experiments to save on sequencing run cost and time, while still permitting deconvolution of the sequencing data, and correlation back to the correct experiment and source biological cells associated therein.

B. Other Reagents.

Various reagents can be used while performing the methods disclosed herein, including buffers (e.g., RT Buffer MX), additives (e.g., Additive RN, RT Additive), and enzymes (e.g., RT Enzyme M, PCR Enzyme K). The enzymes can include, for example, a reverse transcriptase, such as a Moloney murine leukemia virus reverse transcriptase or Maxima reverse transcriptase, or a DNA polymerase, such as the Klenow fragment or PCR Enzyme K (Kapa HiFi), as suitable for the corresponding step in the methods. The buffer can be a buffer that is likewise suitable for the corresponding step in the methods, such as a buffer that support reverse transcriptase (e.g., Moloney murine leukemia virus reverse transcriptase or Maxima reverse transcriptase) activity, and may optionally include MgCl2 (e.g., 100 mM to 150 mM, or about 125 mM). The additives can be selected to support the corresponding step. For example, Additive RN can include 5M betaine.

Sequencing. The methods described herein may be used for 150×150 paired-end reads on an Illumina Miseq (MiSeq Reagent Kit v2 (300 cycle) (Illumina MS-102-2002)). Data analysis first requires demultiplexing the Illumina indexes (if more than one is used), followed by sorting each BCI that identifies each sample, per index. After adapter trimming, etc. the reads may be assembled into the full length TCR (or TCR V(D)J region). The methods described herein may be used for 300×300 paired-end reads.

### IV. KITS

A kit is also provided for use in methods of amplifying paired transcript sequences from single cell such as any of those disclosed herein.

In some embodiments, the kit includes a first RT primer set comprising one or more first RT primers, as described herein. In some embodiments, the kit includes a second RT primer set comprising one or more second RT primers, as described herein. In some embodiments, the kit includes a third RT primer set comprising one or more third RT primers, as described herein.

In some embodiments, the kit includes a first RT primer set, a second RT primer set, and a third RT primer set as disclosed herein.

In some embodiments, the kit includes a set of TCR alpha chain amplification primers, a set of TCR beta chain amplification primers, and a first set of TCR amplification primers.

In some embodiments, the kit includes a first RT primer set, a second RT primer set, a third RT primer set, a set of TCR alpha chain amplification primers, a set of TCR beta chain amplification primers, and a first set of TCR amplification primers as disclosed herein.

In some embodiments, the kit includes no more than six sets of primers.

In some other embodiments, the kit includes a set of shared sequence amplification primers, a set of first gene sequence amplification primers, and a set of second gene sequence amplification primers, as disclosed herein.

Further materials that may be included in the kits include one or more of: dNTP Mix (e.g., Deoxynucleotide (dNTP) Solution Mix (New England Biolabs, N0447L), buffers (e.g., RT Buffer MX), additives (e.g., Additive RN, RT Additive), enzymes (e.g., RT Enzyme M (Thermo Scientific™ Maxima H Minus Reverse Transcriptase (200 U/μL), Fischer Scientific, FEREP0753), PCR Enzyme K (KAPA HiFi HotStart ReadyMix, Roche Diagnostics, KK2602)), a lytic agent (e.g., a lysis buffer), RNase inhibitor (Invitrogen™ RNaseOUT™ Recombinant Ribonuclease Inhibitor, Fischer Scientific, 10-777-019), other reagents disclosed herein, and any combinations thereof. While these particular reagents have been used in this process, the process is not so limited to these specific commercially available materials. Similar materials may be obtained from commercial sources including New England Biolabs, ThemoFisher Scientific, Sigma, Takara, and Roche Diagnostics. One of skill can determine specific conditions under which such substituted reagents may function without departing from the scope of the disclosure. Further, while the experiment describes the use of sequencing adapters and indexes suitable for Illumina sequencing systems, substitution of other adapters and/or indexes may be made by one of skill to similarly determine suitable adaptors, index sequences and conditions other sequencing systems, whether commercially available or proprietary.

In some embodiments, one or more components of the kit is stored at a temperature of about 4° C.

In one embodiment, a kit may contains the reagents and protocols necessary for one or more of the following: (1) purification, RNA from exported T cells, (2) reverse transcription and cDNA amplification, (3) specific amplification of V(D)J regions of human TCR alpha and beta chains. The kit may further comprise protocol and instructions for indexing and sequencing using Illumina MiSeq platform.

In one embodiment, a kit includes a first primer set, a second RT primer set, and a third RT primer set, TCR Primer mix, dNTP Mix, RT Buffer, RT Enzyme, RT Additive, RNase Inhibitor, PCR Enzyme K. The kit may be compatible with Opto T Cell IFNγ assay (Berkeley Lights, Inc).

### V. CELL

In various embodiments, the single cell include an eukaryotic cell, a plant cell, an animal cell, such as a mammalian cell, a reptilian cell, an avian cell, a fish cell, or the like, or prokaryotic cell, bacterial cell, fungal cell, protozoan cell, or the like.

In some embodiments, the single cell is from a cell line. In some embodiments, the single cell is a primary cell isolated from a tissue, such as blood, muscle, cartilage, fat, skin, liver, lung, neural tissue, and the like. In some embodiments, the single cell may be an immune cell, for example a T cell, B cell, NK cell, macrophage, dendritic cell, and the like.

In some embodiments, the single cell may be a cancer cell, such as a melanoma cancer cell, breast cancer cell, neurological cancer cell, etc.

In other embodiments, the single cell may be a stem cell (e.g., embryonic stem cell, induced pluripotent (iPS) stem cell, etc.) or a single cell.

In yet other embodiments, the single cell is an embryo (e.g., a zygote, a 2 to 200 cell embryo, a blastula, etc.), an oocyte, ovum, sperm cell, hybridoma, cultured cell, infected cell, transfected and/or transformed cell, or reporter cell.

A. T Cell Selection.

The capability of T cells to produce specific biological materials (e.g., proteins, such as antibodies or cell surface markers) can be assayed, for example in a microfluidic device. In a specific embodiment T cells are assayed for production of an analyte of interest and selected for based on particular characteristics, for example ability to produce the analyte of interest.

A population of T cells can be obtained through known methods. In some embodiments, peripheral blood mononuclear cells (PBMCs) comprising T cells are isolated from a blood sample, such as a whole blood sample. In some embodiments, T cells are isolated from a solid tumor sample, e.g., a fine needle aspirate or a biopsy. The population of isolated T cells can be enriched for desired cell types or depleted of undesired cell types. Enrichment and depletion can be performed using, e.g., flow cytometry, T cell enrichment columns, antibody-conjugated beads (e.g., magnetic beads), etc., and may use positive or negative selections such as isolating desired cells from a population or removing undesired cells from a population, respectively. For example, magnetic beads conjugated to antibodies to CD45RO can be used to deplete CD45RO+ cells from a population. As another example, magnetic beads conjugated to antibodies to CD45RA can be used to deplete CD45RA+ cells from a population.

In some embodiments, the T cells are enriched for CD3+ T cells. In some embodiments, the one or more T cells are from a population of CD3+ T cells isolated from a peripheral blood sample or a solid tumor sample. In some embodiments, the T cells are enriched for CD3+CD4+ cells (e.g., helper T cells). In some embodiments, the T cells are enriched for CD3+CD8+ cells (e.g., cytotoxic T cells). In some embodiments, the T cells are enriched for both CD3+CD4+ and CD3+CD8+ cells. In some embodiments, the T cells are enriched for CCR7+ cells, e.g., CD3+CCR7+, CD3+CD4+CCR7+, or CD3+CD8+CCR7+ cells. In some embodiments, the T cells are enriched for CD45RA+ cells, e.g., CD3+CD45RA+, CD3+CD4+CD45RA+, or CD3+CD8+CD45RA+ cells. In some embodiments, the T cells are enriched for CD45RO+ cells, e.g., CD3+CD45RO+, CD3+CD4+CD45RO+, or CD3+CD8+CD45RO+ cells.

In some embodiments, the population of T cells (T cells) is enriched for CD3+ T cells. In some embodiments, the population of T cells is enriched for CD3+CD4+ T cells. In some embodiments, the population of T cells is enriched for CD3+CD8+ T cells. In some embodiments, the population of T cells is enriched for CD45RA+ T cells. In some embodiments, the population of T cells is enriched for CD45RO+ T cells. In some embodiments, the population of T cells is enriched for CCR7+ T cells. In some embodiments, the population of T cells is enriched for CD62L+ T cells. In some embodiments, the population of T cells is enriched for CD45RA+/CD7+/CD62L+ T cells. In some embodiments, the population of T cells is enriched for CD45RO+/CD7+/CD62L+ T cells. In some embodiments, the population of T cells is enriched for PD-1+ T cells. In some embodiments, the population of T cells is enriched for CD137+ T cells. In some embodiments, the population of T cells is enriched for PD-1+/CD137+ T cells.

In certain embodiments, at least one T cells can be selected based on cell-surface expression of a marker, such as CD3, CD4, CD8, CD45RO, CD45RA, CCR7, CD62L, PD-1, CD137T, or any combination thereof.

In some embodiments, the at least one selected T cell is selected, at least in part, because its cell surface is CD4+ or CD3+CD4+. In some embodiments, the at least one selected T cell is selected, at least in part, because its cell surface is CD8+ or CD3+CD8+. In some embodiments, the at least one selected T cell is selected, at least in part, because its cell surface is CD45RA+(e.g., CD45RA+CD45RO−). In some embodiments, the at least one selected T cell is selected, at least in part, because its cell surface is CD45RA− (e.g., CD45RA−CD45RO+). In some embodiments, the at least one selected T cell is selected, at least in part, because its cell surface is CCR7+ and/or CD62L+. In some embodiments, the at least one selected T cell is selected, at least in part, because its cell surface is CCR7- and/or CD62L−. In some embodiments, the at least one selected T cell is selected, at least in part, because its cell surface is CD45RA+, CD45RO−, CD45RA+CCR7+, CD45RO−CCR7+, CD45RA+CD45RO−CCR7+, CD45RA+CD62L+, CD45RO−CD62L+, CD45RA+CD45RO−CD62L+, CD45RA+CCR7+CD62L+, CD45RO−CCR7+CD62L+, or CD45RA+CD45RO−CCR7+CD62L+. In some embodiments, the at least one selected T cell is selected, at least in part, because its cell surface is CD45RO+, CD45RA−, CD45RO+CCR7+, CD45RA−CCR7+, CD45RO+CD45RA−CCR7+, CD45RO+CD62L+, CD45RA−CD62L+, CD45RO+CD45RA−CD62L+, CD45RO+CCR7+CD62L+, CD45RA−CCR7+CD62L+, or CD45RO+CD45RA−CCR7+CD62L+. In some embodiments, the at least one selected T cell is selected, at least in part, because its cell surface is CD45RO+CCR7−, CD45RA−CCR7−, CD45RO+CD45RA−CCR7−, CD45RO+CD62L−, CD45RA−CD62L−, CD45RO+CD45RA−CD62L−, CD45RO+CCR7−CD62L−, CD45RA−CCR7−CD62L−, CD45RO+CD45RA−CCR7−CD62L−. In some embodiments, the at least one selected T cell is selected, at least in part, because its cell surface is CD69−. In some embodiments, the at least one selected T cell is selected, at least in part, because its cell surface is PD-1+ and/or CD137+. In some embodiments, the at least one selected T cell is selected, at least in part, because its cell surface is PD-1- and/or CD137−.

In some embodiments, the T cell is selected by a method that comprises contacting the at least one T cell with one or more binding agents (e.g., antibodies, tetramer of peptide-bound MHC complexes, or the like) and selecting at least one T cell based on the presence (or absence) of binding between the one or more binding agents and the at least one T cell. In some embodiments, each of the one or more binding agents comprises a label, such as a fluorophore.

In some embodiments, the T cell is selected based on the proliferation rate of the one or more T cells. In some embodiments, the T cell is selected based on the expression of one or more cytokines, e.g., INFgamma, TNFalpha, IL2, or a combination thereof, by the one or more T cells. In some embodiments, the T cell is selected based on the expression of one or more regulatory T cell markers, such as CTLA4, by the one or more T cells.

In some embodiments, T cells are placed into a well of a wellplate after being exported from the microfluidic device, for example after undergoing expansion. Export can be selective, for example based at least in part on proliferation rate, cytokine expression, expression of regulatory T cell marker(s), or a combination thereof. In some embodiments, a sample of exported T cells is genomically profiled.

Accordingly, in some embodiments, the methods disclosed herein comprise, before placing the T cell in the lysis solution, selecting the T cell based on cell-surface expression of a marker selected from CD3, CD4, CD8, CD45RO, CD45RA, CCR7, CD62L, PD-1, CD137, or any combination thereof. In some embodiments, selecting the T cell comprises performing a positive selection. In some embodiments, selecting the T cell comprises performing a negative selection. In some embodiments, the methods described herein further comprise, before placing the T cell in the lysis solution, selecting the T cell based on expression by the T cell of one or more cytokines. In further embodiments, the one or more cytokines is selected from IFNgamma, TNFalpha, IL2, or a combination thereof. In some embodiments, the single T cell is selected based on being positive for cytokine secretion.

### VI. EXAMPLES

### Example 1: cDNA and TCR Amplification for Live Single T Cell Exports

Materials and Methods

Preparation of antigenic peptide pulsed T2 cells: T2 cells were suspended at 1e6/ml in IMDM (Iscove's Modified Dulbecco Medium) plus 20% FBS (Fetal Bovine Serum) media. SLC45A2 antigenic peptide (40 microgram/mL) and b2-MG (Beta-2 microglobulin, 3 microgram/mL) were added and the T2 cells were incubated for 1 hr at 37° C. under 5% CO2. The cells were spun down at 400 g for 5 min and resuspended at 5e6/mL in the media used for loading (Advanced RPMI, 1× Glutamax, 10% FBS, 10% B27) to the instrument. The media may be other than this media, and may include no animal or, alternatively, no non-human protein.

Preparation of APBs (antigen presenting beads): APBs presenting SLC45A2 antigenic peptide were prepared as described in US20200299351 A1, entitled “Antigen-Presenting Synthetic Surfaces, Covalently Functionalized Surfaces, Activated T Cells, and Uses Thereof” published Sep. 24, 2020, and WO 2019/018801 published Jan. 24, 2019, the entire contents of each of which are herein incorporated by reference. The APBs presenting SLC45A2 antigenic peptide were suspended in the media used for loading to the instrument.

Stimulation of primary human T cells: Human primary T cells were delivered to individual pens, and were stimulated by either 1) 1-3 antigenic peptide pulsed T2 cells, as prepared above, or 2) 1-3 APBs, presenting antigenic peptide, prepared as described above. The T2-stimulated or APB-stimulated T cells were incubated with the respective stimulating cell or bead overnight. The next day, stimulated T cells were exported individually to separate wells in a microwellplate and processed as follows.

Export Plate Preparation: an export plate was set up with a low binding 96-well PCR plate (Eppendorf® twin.tec 96-Well PCR Plate (VWR, 95041-436)). Each well was filled with 10 μL of 2×TCL buffer, and then 30 μL of mineral oil (Sigma, M5904) The plate was sealed, spun at 200 RCF for 1 min (using a desktop centrifuge), and left at room temperature until use. The plate was visually inspected to ensure the 2×TCL buffer (Qiagen, 1070498) was under the mineral oil. The number of plates/wells was prepared to have capacity that exceeds the number of exports desired. The sealed plates not immediately used were stored for up to two days at room temperature. If using a stored plate or if the plate has been bumped, the Export Plate may be spun at 200 RCF for 1 min right before starting export. After exports of the cells were complete, the export plate was spun down at 1000 RCF for 5 min to ensure the exported samples were at the bottom of the wells and placed at −80° C. and stored until ready for downstream processing. For sealing the plate, adhesive PCR plate seals (Thermo Fisher Scientific, AB0558) were used.

RNA Purification and Isolation: Agencourt RNAClean XP Beads may be brought to room temperature. The Export Plate was thawed at room temperature for 5 min and spun at 200 RCF for 30 s. Ten (10) μL of 1× Agencourt RNAClean XP Beads (Beckman Coulter, A63987) were added to each well and gently mixed with a P10 pipette (not by vortexing). Filter tips may be used for RNA handling, as well as for samples for NGS. The Export Plate may be resealed. The Export Plate was incubated for 20 min at room temperature. The Export Plate was placed on the magnetic separator (MagWell™ Magnetic Separator 96 (EdgeBio, 57624) for 5 min. Fresh 80% ethanol was prepared using Nuclease free water (Ambion, AM9937) and 100% pure ethanol (Sigma-Aldrich, E7023). A total of 2.5 ml of 80% ethanol was used for a single bead clean up of a 96-well plate. The supernatant was removed from each well of the export plate by pipetting and discarded. Each well of the Export Plate was washed two times with 50 μL of 80% ethanol (without disturbing the beads during the washing). To ensure all ethanol is removed during each step, the pipette volume may be set to >100 μL, the ethanol may be slowly drawn up until air just enters the tip, and paused for a couple seconds to let residual ethanol run down the walls of the plate; then the residual ethanol may be continued to be drawn up.

The Export Plate was left at room temperature for 2 min to make sure the beads are dry, but not so dry that they start cracking. The beads were resuspended by adding 4 μL of RT Mix 1 (RT Primer 1 (1 μL) (Berkeley Lights, Inc. 520-70033), dNTP mix (1 μL) (Berkeley Lights, Inc. 520-70006), 5M betaine (0.2 μL), Nuclease Free water (1.8 μL)) to all wells with the Eppendorf™ Repeater™ M4 Manual Handheld Pipette Dispenser (4982000322) (Fisher Scientific, 14-278-150), and subsequently mixing with a 10 μL multi-channel pipettor to rinse the beads off the inside walls of the plate.

cDNA Synthesis: the plate was re-sealed and spun at 200 RCF for 10 s. The export plate was incubated in a thermocycler block preheated to 72° C., for 3 min, followed by gradual cooling to 4° C., then the export plate was immediately placed on ice cold block (Universal Medical 81001). With the plate on the cold block, 3 μL of RT Mix 2 (RT Primer 2 (0.5 μL) (Berkeley Lights, Inc. 520-70034), RT Buffer MX (1.3 μL) (Berkeley Lights, Inc, 520-70008), RT Additive (1 μL), 5M betaine (0.1 μL) (Berkeley Lights, Inc, 520-70010), RT Enzyme (0.1 μL) (Berkeley Lights, Inc, 520-70009)) was added to all wells of the export plate, followed by resealing. All reagents and the plate may be kept as cold as possible.

The Export Plate was spun at 200 RCF for 10 sec and placed back into ice cold block immediately for 20 sec. The plate was pulse vortexed using a desktop vortex with 3 inch platform for 5 sec and placed back into ice cold block for 20 sec. The Export Plate was spun at 200 RCF for 10 s. The Export Plate was placed back into ice cold block immediately. The plate may be kept ice cold until the next step. The plate was transferred from the cold block to a thermocycler with a block pre-heated to 42° C., and incubated for (1) 42° C. for 90 min, (2) 10 cycles of 50° C. for 2 min/42° C. for 2 min, (3) heat inactivate at 85° C. for 5 min, and (4) hold at 4° C. until the next step (the plate may be left at 4° C. overnight).

Total cDNA Amplification for TCR HC/LC: 18 μL of RT Mix 3 (RT Primer 3 (0.25 μL) (Berkeley Lights, Inc. 520-70035), PCR Enzyme K (Kapa HiFi HotStart ready mix, Roche Diagnostics, (12.5 μL), Nuclease free water (5.25 μL)) was added to all wells of the export plate. The plate was resealed, and pulse vortexed for total of no more than 5 sec and spun at 200 RCF for 10 s. The plate was incubated (1) at 98° C. for 3 min, (2) 20 cycles of 98° C. for 20 s/65° C. for 30 s/72° C. for 6 min, (3) 72° C. for 10 min, and (4) hold at 4° C. until the next step (the plate may be left at 4° C. for up to 3 days). A PCR Thermocycler was used for the PCR amplification.

The beads may be brought to room temperature before use. 20 μL of Agencourt AMPure XP Beads (Beckman Coulter, A63881) was added to each well. The plate may be sealed at this point. The plate was pulse vortexed until the beads were completely suspended (˜10 sec.). The Export Plate was spun at 200 RCF for 10 s, to ensure there was no liquid on the seal, and incubated for 10 min. at room temperature.

The Export Plate was placed on the magnetic separator for 5 min. The MagWell™ magnetic Separator 96 may be used for this step. Fresh 80% ethanol was prepared. RNase free water and 100% pure ethanol may be used for the preparation. A total of 2.5 ml of 80% ethanol was used for a single bead cleanup of a 96-well plate. After the supernatant was removed from each well of the export plate by pipetting and discarded, each well of the export plate was washed two times with 50 μL of 80% ethanol (without disturbing the beads during the washing). To ensure all ethanol is removed during each step, the pipette volume may be set up to >100 μL, the ethanol may be slowly drawn up until air just enters the tip, and paused for a couple seconds to let residual ethanol run down the walls of the plate; then the residual ethanol may be continued to be drawn up. The export plate was left at room temperature for 2 min to dry the beads (but not too dry that they start cracking). The cDNA was eluted from the beads by adding 30 μL of Nuclease Free water. The cDNA quality was checked by a high-sensitivity BioAnalyzer (or by qPCR of relevant genes (Actin B or TCR)).

The following steps are performed to amplify TCRs from cDNA, index with Illumina Nextera indexes and sequence on the MiSeq platform. Alternatively, any other methods of performing amplicon sequencing as commonly known in the art may be performed.

TCR amplification: 10 μL TCR amplification mix 1 was prepared with PCR Enzyme K (5 μL), TCR Amp primer mix (0.4 μL) (Berkeley Lights, Inc. 520-70036), Nuclease Free water (3.6 μL), and the cDNA (1 μL) obtained as described above, for each well. The plate was pulse vortexed for 5 sec and spun at 200 RCF for 10 s. The plate was incubated as follows: (1) at 96° C. for 3 min; (2) 23 cycles of 98° C. for 20 sec/70° C. for 30 s/72° C. for 20 s, (3) 72° C. for 5 min, and (4) at 4° C. until the next step (the plate may be left at 4° C. for up to 3 days). The cycle number can be increased if cDNA amplification is sub-optimal. Increased cycle numbers also may increase the fraction of TCRs with PCR based mutations.

TCR indexing: 10 μL of TCR amplification mix 2 was prepared with PCR Enzyme K (5 μL), TCR Index (N7XX and S5XX indexes are pre-mixed) (from Illumina, A (FC-131-2001), B (FC-131-2002), C (FC-131-2003), D (FC-131-2004)) (0.5 μL), Nuclease Free water (4 μL), Amplified TCR (0.5 μL). To add Illumina Nextera indexes to amplified TCRs, 10 μL TCR amplification mix 2 per well was added in a new 96-well plate. Each well of TCRs had a unique index pair. Amplified TCRs may be transferred directly to the indexing reaction without purification.

TCR Pooling Indexed TCRs may be stored at −20° C. or pooled for sequencing. Pooling may be performed in a manner known in the art; for example, 2-4 μL may be removed from each PCR reaction using an 8-channel pipette, and each aliquot may be combined in 8 clean PCR tubes/wells, using a P200 pipette to combine pooled TCRs from the 8 tubes/wells into a single 1.5 mL tube.

Indexed TCR clean up A known volume (typically 50 μL) of pooled TCRs was transferred to a PCR tube/well plate (or 1.5 mL tube). 0.8 volumes of room temperature AMPure Beads was added and vortexed. The indexed TCRs were washed and removed from the beads by performing the following: the Export Plate was placed on the magnetic separator for 5 min. After the supernatant was removed from each well of the export plate by pipetting and discard, each well of the export plate was washed two times with 50 μL of 80% ethanol (without disturbing the beads during the washing). The export plate was left at room temperature for 2 min to dry the beads (but not too dry that they start cracking). The cDNA was eluted from the beads by adding 30 μL of Nuclease Free water.

DNA Concentration: The concentration of the library was estimated using Qubit. This concentration was used to load the Bioanalyzer with the appropriate amount of library. The pooled, indexed TCRs was run on a HS Bioanalyzer chip prior to sequencing to determine the average size of the library (about 700 bp) and ensure all primers have been removed from the sample.

Sequencing: the library concentration was calculated using the following equation along with your Qubit concentration reading and average library size determined using BioAnalyzer:

$$[ \frac{ng}{μ L} ] = \frac{[ nM ] \times ( average size bp ) \times 650}{{10}^{6}}$$

The MiSeq sequencing kit (MiSeq™ Reagent Kit v2 (300-cycles) (15033412) or MiSeg™ Reagent Micro Kit v2 (300-cycles) (15036523)) was chosen based on the number of samples being sequenced and the desired sequence depth. 2000-10000 reads per sample may be used. The library was diluted and prepared according to the manufacturer' protocol. For a 300 base read kit, Read 1 should be 50 base reads and Read 2 should be 250 base reads to capture CDR2 to CDR3 sequences. Index read lengths are 8 base reads. For a 600 base read kit, 300×300 should capture the entire V(D)J sequence.

TCR Analysis: MiXCR was used to analyze TCR sequencing FASTQ files.

Results:

TCR Sequence Recovery

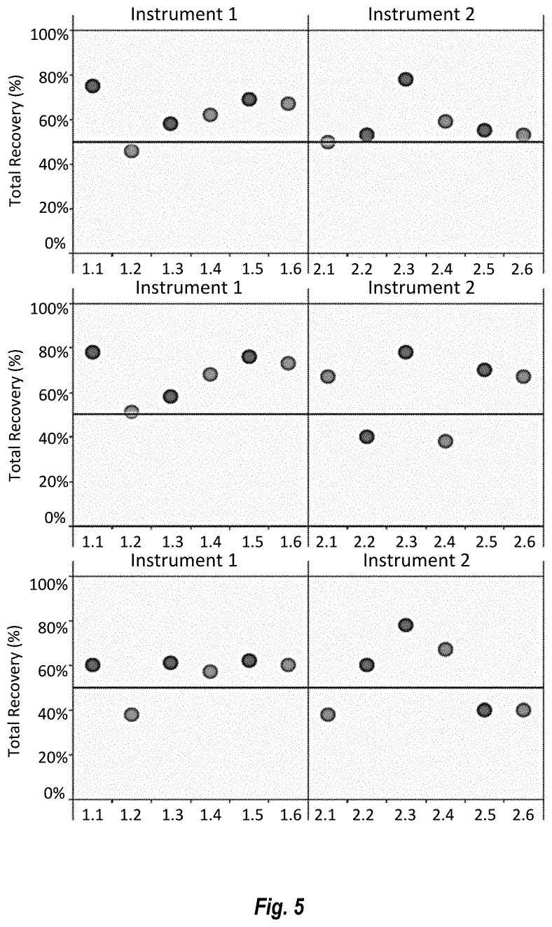

The acceptance criteria for TCR chain (alpha and beta) recovery from successfully unpenned T cells was >50%, which was achieved on average over 12 separate workflows, six performed on Instrument 1 (Experiment 1.1-1.6) and six on Instrument 2 (Experiment 2.1-2.6)(FIG. 5). Individually, only one plate processed (Experiment 1.2 on Instrument 1) fell just below 50% recovery. This plate had the highest number of unpenning failures out of all experiments. High frequencies of unpenning failures can be indicative of poor cell health, which might have reduced the overall success of cDNA/TCR recovery in Experiment 1.2. Similarly, TCR recovery was higher from cells stimulated by T2 target cells vs. APBs, but the differences were also not statistically significant). Applying a 2-tailed, paired T-test, there were no significant differences between Instrument 1 and instrument 2 (P-value=0.48), or between T2-stimulated or bead-stimulated unloads (P-value=0.14), or any of the four conditions (Instrument 1+ T2 Stim, Instrument 1+APB Stim, Instrument 2+ T2 Stim, Instrument 2+APB Stim).

TCR Pairing and Clonality

TCR sequencing results were analyzed using an R script modified from live human primary T cell export and OptoSeq BCR workflows (Berkley Lights, Inc, 750-01001). The script removed all TCRs sequenced from T2 cells and clones with very low read counts (<5% of clonal fraction). Reads of TCR chains only differing by sequencing/PCR-based point mutations were collapsed into a single chain based on its read abundance. The alpha and beta TCR chains (TCR clones) associated with each export were analyzed and curated according to the different Donors A to C, showing the clonal variation across all experiments (Tables 1A-1C).

Clonal clustering was distinct and varied among the three donors, with each pair of CDR3 sequences being unique among the three donors. The rows in bold indicate TCR clones that have the same alpha and beta variable domains. The rows in bold and in italic indicate TCR clones that share beta chains with the same CDR3 sequence. Variable domain pairs (Trav17+Trbv13) were shared among donors (see Tables 1A-1C, in bold). Only one specific (CDR3 sequence) TCR beta chain was sequenced from more than one donor (Tables 1A-1C, in bold and italic). However, both clones were paired with distinct alpha chains. The distinct clonal pairs clustered to each donor indicate that the methods described herein can reliably yield distinct TCR clones (of varying abundance) over multiple experiments, with little ambiguity.

TABLE 1A
Summary of all unique TCR clones and their representation from Donor A

| | | Alpha | | | | | | |
|---|---|---|---|---|---|---|---|---|
| TCR | | variable | | Beta Variable | | | Count | |
| Clone # | Alpha chain | domain (V) | Beta chain | domain (V) | Count | Fraction | (+singles) | Fraction |
| A-1 | CAMSEWVTGTASK | TRAV12-3 | CATSDRGSPEAFF | TRBV24-1 | 24 | 0.27 | 25 | 0.24 |
| | LTF (SEQ ID NO: 11) | | (SEQ ID NO: 51) | | | | | |
| A-2 | CATDSIHTNAGKST | TRAV17*0 | CASSRTSNGEQFF | TRBV5-6* | 15 | 0.17 | 15 | 0.15 |
| | F (SEQ ID NO: 12) | | (SEQ ID NO: 52) | | | | | |
| A-3 | CAMSASSGAGSYQ | TRAV12-3 | CATSDRGSPEAFF | TRBV24-1 | 15 | 0.17 | 22 | 0.21 |
| | LTF (SEQ ID NO: 13) | | (SEQ ID NO: 51) | | | | | |
| A-4 | CATDWTGGFKTIF | TRAV17*0 | CASRPQGDGYTF | TRBV5-6* | 13 | 0.15 | 15 | 0.15 |
| | (SEQ ID NO: 14) | | (SEQ ID NO: 53) | | | | | |
| A-5 | CALNTGFQKLVF | TRAV9-2* | CASSYSLGEDGDTQ | TRBV6-5* | 11 | 0.12 | 14 | 0.14 |
| | (SEQ ID NO: 15) | | YF | | | | | |
| | | | (SEQ ID NO: 54) | | | | | |
| A-6 | CAMTNDYKLSF | TRAV12-3 | CASSLSYEQYF | TRBV11-1 | 6 | 0.07 | 6 | 0.06 |
| | (SEQ ID NO: 16) | | (SEQ ID NO: 55) | | | | | |
| A-7 | CATDLGIIPPVTNA | TRAV17*0 | CASSGSGTYEQYF | TRBV13*0 | 2 | 0.02 | 3 | 0.03 |
| | GKSTF (SEQ ID NO: | | (SEQ ID NO: 56) | | | | | |
| | 17) | | | | | | | |
| A-8 | CAPPRFRVSNFGNE | TRAV16*0 | CASIPENSGNTGELF | TRBV7-9* | 1 | 0.01 | 1 | 0.01 |
| | KLTF (SEQ ID NO: | | F | | | | | |
| | 18) | | (SEQ ID NO: 57) | | | | | |
| A-9 | CATDSIHTNAGKST | TRAV17*0 | CASSYSLGEDGDTQ | TRBV6-5* | 1 | 0.01 | 1 | 0.01 |
| | F(SEQ ID NO: 12) | | YF | | | | | |
| | | | (SEQ ID NO: 54) | | | | | |
| A-10 | [symbol] [symbol] | TRAV23/D | [symbol] [symbol] | TRBV5-6* | 1 | 0.01 | 1 | 0.01 |
| | (SEQ ID NO: 19) | | (SEQ ID NO: 58) | | | | | |
| | Total | | | | 89 | | 103 | |

TABLE 1B
Summary of all unique TCR clones and their representation from Donor B

| | | Alpha | | | | | | |
|---|---|---|---|---|---|---|---|---|
| | | variable | | Beta | | | | |
| TCR | | domain | | Variable | | | Count | |
| Clone # | Alpha chain | (V) | Beta chain | domain (V) | Count | Fraction | (+singles) | Fraction |
| B-1 | CAVQANNNAGNMLTF | TRAV20*0 | CASSLGQGYYGYTF | TRBV13*0 | 73 | 0.58 | 77 | 0.53 |
| | (SEQ ID NO: 20) | | (SEQ ID NO: 59) | | | | | |
| B-2 | CAVPGADQGAQKLVF | TRAV39*0 | CASSLGPSSPSTDTQYF | TRBV13*0 | 21 | 0.17 | 28 | 0.19 |
| | (SEQ ID NO: 21) | | (SEQ ID NO: 60) | | | | | |
| B-3 | CAFTSGTYKYIF | TRAV24*O | CASSKSGQGSYEQYF | TRBV13*0 | 11 | 0.09 | 12 | 0.08 |
| | (SEQ ID NO: 22) | | (SEQ ID NO: 61) | | | | | |
| B-4 | CAVATNSGNTPLVF | TRAV8-3* | CSTPMWQGEGTEAFF | TRBV20-1 | 11 | 0.09 | 16 | 0.11 |
| | (SEQ ID NO: 23) | | (SEQ ID NO: 62) | | | | | |
| B-5 | CALGEQGGSEKLVF | TRDVl*01 | CASSLATTARYEQYF | TRBV13*0 | 4 | 0.03 | 5 | 0.03 |
| | (SEQ ID NO: 24) | | (SEQ ID NO: 63) | | | | | |
| B-6 | CAGARNNDMRF | TRAV27*0 | CASSLGGYPNGYTF | TRBV5-6* | 4 | 0.03 | 6 | 0.04 |
| | (SEQ ID NO: 25) | | (SEQ ID NO: 64) | | | | | |
| B-7 | CATDATSGTYKYIF | TRAV17* | CASSPGLGETQYF | TRBV13*0 | 1 | 0.01 | 2 | 0.01 |
| | (SEQ ID NO: 26) | 0 | (SEQ ID NO: 65) | | | | | |
| | Total | | | | 125 | | 146 | |

TABLE 1C
Summary of all unique TCR clones and their representation from Donor C

| | | Alpha | | Beta | | | | |
|---|---|---|---|---|---|---|---|---|
| TCR | | variable | | Variable | | | | |
| Clone | | domain | | domain | | | Count | |
| # | Alpha | (V) | Beta | (V) | Count | Fraction | (+singles) | Fraction |
| C-1 | [symbol] [symbol] | TRAV23/ | [symbol] [symbol] | TRBV5-6* | 20 | 0.15 | 22 | 0.14 |
| | (SEQ ID NO: 27) | D | (SEQ ID NO: 66) | | | | | |
| C-2 | CIVRDSNYQLIW | TRAV26- | CAWSTHNVEQYF | TRBV30*0 | 15 | 0.11 | 17 | 0.11 |
| | (SEQ ID NO: 28) | 1 | (SEQ ID NO: 67) | | | | | |
| C-3 | CAGGQSWDNYGQNF | TRAV35*0 | CASSLTGSHEQYF | TRBV13*0 | 15 | 0.11 | 21 | 0.13 |
| | VF | | (SEQ ID NO: 68) | | | | | |
| | (SEQ ID NO: 29) | | | | | | | |
| C-4 | CAVVSGYALNF | TRAV8- | CASSSGQGQPQHF | TRBV13*0 | 13 | 0.10 | 23 | 0.14 |
| | (SEQ ID NO: 30) | 3* | (SEQ ID NO: 69) | | | | | |
| C-5 | CAPRGGGADGLTF | TRAV13- | CASSLDSRGGNTEAFF | TRBV11-2 | 12 | 0.09 | 15 | 0.09 |
| | (SEQ ID NO: 31) | 1 | (SEQ ID NO: 70) | | | | | |
| C-6 | CAAASGGSYIPTF | TRAV12- | CASSYLDSLNTEAFF | TRBV2*01 | 10 | 0.08 | 10 | 0.06 |
| | (SEQ ID NO: 32) | 2 | (SEQ ID NO: 71) | | | | | |
| C-7 | CAVEGLTTDSWGKFQ | TRAV22*0 | CASSYGPNYGYTF | TRBV6-6* | 9 | 0.07 | 10 | 0.06 |
| | F | | (SEQ ID NO: 72) | | | | | |
| | (SEQ ID NO: 33) | | | | | | | |
| C-8 | CLVGSSNTGKLIF | TRAV4*01 | CASSFTGHRTYEQYF | TRBV13*0 | 8 | 0.06 | 8 | 0.05 |
| | (SEQ ID NO: 34) | | (SEQ ID NO: 73) | | | | | |
| C-9 * | CACPSGGYQKVTF | TRAV24* | CASSFLDSPLNEKLFF | TRBV28*0 | 6 | 0.05 | 6 | 0.04 |
| | (SEQ ID NO: 35) | 0 | (SEQ ID NO: 74) | | | | | |
| C-10 | CAVRDEGSARQLTF | TRAV3*01 | CASSSASTGTGFLETQYF | TRBV13*0 | 4 | 0.03 | 4 | 0.02 |
| | (SEQ ID NO: 36) | | (SEQ ID NO: 75) | | | | | |
| c-11 | CALSVYNAGGTSYGKLTF | TRAV19*0 | CASTVAGVGYEQYF | TRBV13*0 | 4 | 0.03 | 7 | 0.04 |
| | (SEQ ID NO: 37) | | (SEQ ID NO: 76) | | | | | |
| C-12 | CAAWEGDDKIIF | TRAV13-1 | CAWSVGGIGGYTF | TRBV30*0 | 3 | 0.02 | 3 | 0.02 |
| | (SEQ ID NO: 38) | | (SEQ ID NO: 77) | | | | | |
| C-13 | CALSEAFTDSWGKFQF | TRAV19*O | C CASSLGLAGDDLSTDTQYF | TRBV28*0 | 3 | 0.02 | 5 | 0.03 |
| | (SEQ ID NO: 39) | | (SEQ ID NO: 78) | | | | | |
| C-14 | CAGQKTSYDKVIF | TRAV22*0 | CASSGRWQ_LAPTSSTS | TRBV28*0 | 2 | 0.02 | 2 | 0.01 |
| | (SEQ ID NO: 40) | | (SEQ ID NO: 79) | | | | | |
| C-15 | CAARSGGYQKVTF | TRAV13-1 | CASSEAQRWGNTEAFF | TRBV6-1* | 1 | 0.01 | 1 | 0.01 |
| | (SEQ ID NO: 41) | | (SEQ ID NO: 80) | | | | | |
| C-16 | CAVSGASYDKVIF | TRAV8-4* | CASSWGGGELFF | TRBV5-8* | 1 | 0.01 | 1 | 0.01 |
| | (SEQ ID NO: 42) | | (SEQ ID NO: 81) | | | | | |
| C-17 ** | CAGGGSQGNLIF | TRAV12-2 | CASSGDSNTEAFF | TRBV13*0 | 1 | 0.01 | 1 | 0.01 |
| | (SEQ ID NO: 43) | | (SEQ ID NO: 82) | | | | | |
| C-18 | CAAVSNDYKLSF | TRAV13-1 | CAASVKSNYQLIW | TRAV29/D | 1 | 0.01 | 1 | 0.01 |
| *** | (SEQ ID NO: 44) | | (SEQ ID NO: 83) | | | | | |
| C-19 | CATDTHNNDMRF | TRAV17*0 | CASSLGRESGYTF | TRBV13*0 | 1 | 0.01 | 1 | 0.01 |
| | (SEQ ID NO: 45) | | (SEQ ID NO: 84) | | | | | |
| C-20 | CAVFSNSGYALNF | TRAV22*0 | CASSYGRGTVPLHF | TRBV6-5* | 1 | 0.01 | 1 | 0.01 |
| | (SEQ ID NO: 46) | | (SEQ ID NO: 85) | | | | | |
| C-21 | CALSEAYGQNFVF | TRAV19*0 | CASKQTSGRYNEQFF | TRBV6-5* | 1 | 0.01 | 1 | 0.01 |
| | (SEQ ID NO: 47) | | (SEQ ID NO: 86) | | | | | |
| C-22 | CAVATINNAGNMLTF | TRAV8-3* | CAWSVGGGTEAFF | TRBV30*0 | 1 | 0.01 | 1 | 0.01 |
| | (SEQ ID NO: 48) | | (SEQ ID NO: 87) | | | | | |
| | Total | | | | 132 | | 161 | |

* C9: the 3^{rd} chain having CAVNGRRQAKSTF (TRAV12-2)(SEQ ID NO: 93)
** C17: the 3^{rd} chain having CAFCGGNQFYF (TRAV24*0)(SEQ ID NO: 94)
*** C18: the 3^{rd} chain having CASSLGLGQPQHF (TRBV13*0)(SEQ ID NO: 95)

Process Variability

Experiments were conducted to test the sensitivity or robustness of the workflow according to the methods described herein to small variations in reagent formulation and/or potential errors in the composition of master mixes (e.g., due to pipetting error). Experiments were performed by systematically increasing or decreasing the concentration of each reagent in the workflow by 10% v/v (replacing with water), using FACS-sorted single T cells as the input for cDNA synthesis. Sensitivity was determined using qPCR and the distribution of Ct values for TCR cDNAs. Standard (unmodified) conditions were run as a positive control for every well plate processed. It was determined that that a +/−10% v/v deviation in reagent/master mix composition causes no apparent change to TCR yield when prepared from single T cells.

Various embodiments are described herein with reference to the figures. It should be noted that the figures are not drawn to scale and that elements of similar structures or functions are represented by like reference numerals throughout the figures. It should also be noted that the figures are only intended to facilitate the description of the embodiment and are not intended as an exhaustive description of the disclosure or as a limitation on the scope of the disclosed inventions, which is defined only by the appended claims and their equivalents. In addition, the respective illustrated embodiments need not each have all the aspects or advantages of features described herein. An aspect or an advantage described in conjunction with a particular embodiment of the disclosure is not necessarily limited to that embodiment and can be practiced in any other embodiments even if not so illustrated. The specification and drawings are, accordingly, to be regarded in an illustrative rather than restrictive sense.

In addition to any previously indicated modification, numerous other variations and alternative arrangements may be devised by those skilled in the art without departing from the spirit and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation, and use may be made without departing from the principles and concepts set forth herein. Also, as used herein, the examples and embodiments, in all respects, are meant to be illustrative only and should not be construed to be limiting in any manner. Furthermore, where reference is made herein to a list of elements (e.g., elements a, b, c), such reference is intended to include any one of the listed elements by itself, any combination of less than all of the listed elements, and/or a combination of all of the listed elements. Also, as used herein, the terms a, an, and one may each be interchangeable with the terms at least one and one or more. It should also be noted, that while the term step is used herein, that term may be used to simply draw attention to different portions of the described methods and is not meant to delineate a starting point or a stopping point for any portion of the methods, or to be limiting in any other way.

### VII. DESCRIPTION OF SEQUENCES

The following provides a listing of certain sequences referenced herein.

| SEQ | | |
|---|---|---|
| ID | | |
| NO. | Sequence | Description |
| 1 | 5′- AAGCAGTGGTATCAACGCAGAGTACTTTTTTTT | Exemplary First |
| | TTTTTTTTTTTTTTTTTTTTTTVN - 3′ | RT Primer Set/ |
| | | RT Primer 1 |
| 2 | 5′- GTGGTATCAACGCAGAGTACACGACGCTCTTC | Exemplary |
| | CGATCTrGrGrG - 3′ | Second RT |
| | | Primer / TSO |
| 3 | 5′- AAGCAGTGGTATCAACGCAGAGT - 3′ | Exemplary Third |
| | | Primer |
| 4 | 5′- TCGTCGGCAGCGTCAGATGTGTATAAGAGACA | Exemplary First |
| | GTACACGACGCTCTTCCGATCTG - 3′ | TCR |
| | | Amplification |
| | | Primer |
| | | Underlined |
| | | indicates a |
| | | shared forward |
| | | TCR |
| | | complement |
| | | subsequence |
| 5 | 5′- GTCTCGTGGGCTCGGAGATGTGTATAAGAGACA | Exemplary TCR |
| | GACGGCAGGGTCAGGGTTCTG - 3′ | Alpha |
| | | Amplification |
| | | Primer |
| | | Underlined |
| | | indicatesa |
| | | subsequence |
| | | complementary |
| | | to a sequence of |
| | | the TCR alpha |
| | | chain (TRAC) |
| 6 | 5′- GTCTCGTGGGCTCGGAGATGTGTATAAGAGACA | Exemplary TCR |
| | GGCGACCTCGGGTGGGAACAC - 3′ | Beta |
| | | Amplification |
| | | Primer |
| | | Underlined a |
| | | subsequence |
| | | complementary |
| | | to a sequence of |
| | | the TCR beta |
| | | chain sequences |
| | | (TRBC1/TRBC2) |
| 7 | TTGAAACCCT TCAAAGGCAG AGACTTGTCC | Exemplary TCR |
| | AGCCTAACCT GCCTGCTGCT CCTAGCTCCT | alpha chain |
| | GAGGCTCAGG GCCCTTGGCT TCTGTCCGCT | (TRAC) |
| | CTGCTCAGGG CCCTCCAGCG TGGCCACTGC | sequence |
| | TCAGCCATGC TCCTGCTGCT CGTCCCAGTG | |
| | CTCGAGGTGA TTTTTACCCT GGGAGGAACC | |
| | AGAGCCCAGT CGGTGACCCA GCTTGGCAGC | |
| | CACGTCTCTG TCTCTGAAGG AGCCCTGGTT | |
| | CTGCTGAGGT GCAACTACTC ATCGTCTGTT | |
| | CCACCATATC TCTTCTGGTA TGTGCAATAC | |
| | CCCAACCAAG GACTCCAGCT TCTCCTGAAG | |
| | TACACATCAG CGGCCACCCT GGTTAAAGGC | |
| | ATCAACGGTT TTGAGGCTGA ATTTAAGAAG | |
| | AGTGAAACCT CCTTCCACCT GACGAAACCC | |
| | TCAGCCCATA TGAGCGACGC GGCTGAGTAC | |
| | TTCTGTGCTG TGAGTGATCT CGAACCGAAC | |
| | AGCAGTGCTT CCAAGATAAT CTTTGGATCA | |
| | GGGACCAGAC TCAGCATCCG GCCAAATATC | |
| | CAGAACCCTG ACCCTGCCGT GTACCAGCTG | |
| | AGAGACTCTA AATCCAGTGA CAAGTCTGTC | |
| | TGCCTATTCA CCGATTTTGA TTCTCAAACA | |
| | AATGTGTCAC AAAGTAAGGA TTCTGATGTG | |
| | TATATCACAG ACAAAACTGT GCTAGACATG | |
| | AGGTCTATGG ACTTCAAGAG CAACAGTGCT | |
| | GTGGCCTGGA GCAACAAATC TGACTTTGCA | |
| | TGTGCAAACG CCTTCAACAA CAGCATTATT | |
| | CCAGAAGACA CCTTCTTCCC CAGCCCAGAA | |
| | AGTTCCTGTG ATGTCAAGCT GGTCGAGAAA | |
| | AGCTTTGAAA CAGATACGAA CCTAAACTTT | |
| | CAAAACCTGT CAGTGATTGG GTTCCGAATC | |
| | CTCCTCCTGA AAGTGGCCGG GTTTAATCTG | |
| | CTCATGACGC TGCGGCTGTG GTCCAGCTGA | |
| 8 | GGCTCCAGGC TGCTCTGTTG GGTGCTGCTT | Exemplary TCR |
| | TGTCTCCTGG GAGCAGGCCC AGTAAAGGCT | beta chain 1 |
| | GGAGTCACTC AAACTCCAAG ATATCTGATC | (TRBC1) |
| | AAAACGAGAG GACAGCAAGT GACACTGAGC | sequence |
| | TGCTCCCCTA TCTCTGGGCA TAGGAGTGTA | |
| | TCCTGGTACC AACAGACCCC AGGACAGGGC | |
| | CTTCAGTTCC TCTTTGAATA CTTCAGTGAG | |
| | ACACAGAGAA ACAAAGGAAA CTTCCCTGGT | |
| | CGATTCTCAG GGCGCCAGTT CTCTAACTCT | |
| | CGCTCTGAGA TGAATGTGAG CACCTTGGAG | |
| | CTGGGGGACT CGGCCCTTTA TCTTTGCGCC | |
| | AGCAGCCGGA CAGGGGGCGT AAAGAATTCA | |
| | CCCCTCCACT TTGGGAACGG GACCAGGCTC | |
| | ACTGTGACAG AGGACCTGAA CAAGGTGTTC | |
| | CCACCCGAGG TCGCTGTGTT TGAGCCATCA | |
| | GAAGCAGAGA TCTCCCACAC CCAAAAGGCC | |
| | ACACTGGTGT GCCTGGCCAC AGGCTTCTTC | |
| | CCTGACCACG TGGAGCTGAG CTGGTGGGTG | |
| | AATGGGAAGG AGGTGCACAG TGGGGTCAGC | |
| | ACGGACCCGC AGCCCCTCAA GGAGCAGCCC | |
| | GCCCTCAATG ACTCCAGATA CTGCCTGAGC | |
| | AGCCGCCTGA GGGTCTCGGC CACCTTCTGG | |
| | CAGAACCCCC GCAACCACTT CCGCTGTCAA | |
| | GTCCAGTTCT ACGGGCTCTC GGAGAATGAC | |
| | GAGTGGACCC AGGATAGGGC CAAACCCGTC | |
| | ACCCAGATCG TCAGCGCCGA GGCCTGGGGT | |
| | AGAGCAGACT GTGGCTTTAC CTCGGTGTCC | |
| | TACCAGCAAG GGGTCCTGTC TGCCACCATC | |
| | CTCTATGAGA TCCTGCTAGG GAAGGCCACC | |
| | CTGTATGCTG TGCTGGTCAG CGCCCTTGTG | |
| | TTGATGGCCA TGGTCAAGAG AAAGGATTTC TGA | |
| 9 | GACCTGAAAA ACGTGTTCCC ACCCAAGGTC | Exemplary TCR |
| | GCTGTGTTTG AGCCATCAGA AGCAGAGATC | beta chain 2 |
| | TCCCACACCC AAAAGGCCAC ACTGGTGTGC | (TRBC2) |
| | CTGGCCACAG GCTTCTACCC CGACCACGTG | sequence |
| | GAGCTGAGCT GGTGGGTGAA TGGGAAGGAG | |
| | GTGCACAGTG GGGTCAGCAC AGACCCGCAG | |
| | CCCCTCAAGG AGCAGCCCGC CCTCAATGAC | |
| | TCCAGATACT GCCTGAGCAG CCGCCTGAGG | |
| | GTCTCGGCCA CCTTCTGGCA GAACCCCCGC | |
| | AACCACTTCC GCTGTCAAGT CCAGTTCTAC | |
| | GGGCTCTCGG AGAATGACGA GTGGACCCAG | |
| | GATAGGGCCA AACCTGTCAC CCAGATCGTC | |
| | AGCGCCGAGG CCTGGGGTAG AGCAGACTGT | |
| | GGCTTCACCT CCGAGTCTTA CCAGCAAGGG | |
| | GTCCTGTCTG CCACCATCCT CTATGAGATC | |
| | TTGCTAGGGA AGGCCACCTT GTATGCCGTG | |
| | CTGGTCAGTG CCCTCGTGCT GATGGCCATG | |
| | GTCAAGAGAA AGGATTCCAG AGGCTAG | |
| 96 | 5′-TACACGACGCTCTTCCGATCTG -3′ | Exemplary |
| | | shared forward |
| | | TCR |
| | | complement |
| | | subsequence of |
| | | the first set of |
| | | TCR |
| | | amplification |
| | | primer |
| 97 | 5′- ACGGCAGGGTCAGGGTTCTG -3′ | Exemplary |
| | | subsequence |
| | | complementary |
| | | to a sequence of |
| | | the TCR alpha |
| | | chain (TRAC) |
| 98 | 5′- GCGACCTCGGGTGGGAACAC - 3′ | Exemplary TCR |
| | | subsequence |
| | | complementary |
| | | to a sequence of |
| | | the TCR beta |
| | | chain sequences |
| | | (TRBC1/TRBC2) |
| 99 | 5′- CAGAACCCTGACCCTGCCGT -3′ | Reverse |
| | | complementary |
| | | sequence to SEQ |
| | | ID NO: 97 |
| 100 | 5′- GTGTTCCCACCCGAGGTCGC - 3′ | Reverse |
| | | complementary |
| | | sequence to SEQ |
| | | ID NO: 98 |

1. A method for paired amplification of alpha and beta T cell receptor (TCR) sequences from a single T cell, the method comprising:
placing a single T cell into a cell lysis solution to provide a T cell lysate comprising RNA;
generating first strand cDNA from the RNA;
amplifying the first strand cDNA to provide amplified cDNA; and
amplifying the alpha and beta TCR sequences from the amplified cDNA in a single reaction,
wherein the alpha and beta TCR sequences are amplified using three sets of TCR amplification primers.

2. The method of claim 1, wherein generating first strand cDNA comprises: performing a reverse transcription (RT) reaction by contacting the RNA with a first RT primer set, a second RT primer set, and an enzyme having reverse transcriptase activity, for a first period of time and under conditions that generate first strand cDNA.

3. The method of claim 2, wherein the first RT primer set comprises one or more first RT primers, each first RT primer comprising a poly-T subsequence.

4. The method of claim 3, wherein each first RT primer of the first RT primer set comprises a 3′ terminal VN subsequence, wherein V is any nucleotide selected from A, C, and G, wherein N is any nucleotide selected from A, C, G, and T, and wherein the poly-T subsequence is located 5′ to the 3′ terminal VN subsequence.

5. The method of claim 2, wherein each first RT primer of the first RT primer set comprises a first amplification priming subsequence.

6. The method of claim 2, wherein the second RT primer set comprises one or more template switching oligos (TSOs), wherein each TSO comprises a 3′ terminal ribonucleotide subsequence.

7. The method of claim 6, wherein the second RT primer set comprises a single TSO.

8. The method of claim 6, wherein each TSO of the second RT primer set comprises a second amplification priming subsequence located 5′ to the 3′ terminal ribonucleotide subsequence.

9. The method of claim 8, wherein the first amplification priming subsequence of the first RT primer set and the second amplification priming subsequence of the second RT primer set have substantially identical nucleotide sequences.

10. The method of claim 1, wherein amplifying the first strand cDNA comprises contacting the first strand cDNA with a third primer set comprising one or more third primers, wherein each third primer of the third primer set comprises a third amplification priming subsequence, for a second period of time and under conditions that generate amplified cDNA.

11. The method of claim 10, wherein the third primer set comprises a single primer.

12. The method of claim 11, wherein the first amplification priming subsequence of the first RT primer set, the second amplification priming subsequence of the second RT primer set, and the third amplification priming subsequence of the third primer set all have substantially identical nucleotide sequences.

13. The method of claim 1, wherein amplifying the alpha and beta TCR sequences comprises contacting the amplified cDNA with the three sets of TCR amplification primers for a third period of time and under conditions that provide amplification of paired alpha and beta T cell receptor (TCR) sequences from the amplified cDNA, wherein the three sets of TCR amplification primers comprise a first set of TCR amplification primers, a set of TCR alpha chain amplification primers, and a set of TCR beta chain amplification primers.

14. The method of claim 13, wherein each primer of the set of TCR alpha chain amplification primers comprises a TCR alpha chain complement subsequence that is substantially identical to a complement sequence of a constant region sequence of a TCR alpha chain.

15. (canceled)

16. The method of claim 13, wherein the set of TCR alpha chain amplification primers comprises a single TCR alpha chain amplification primer.

17. The method of claim 13, wherein each primer of the set of TCR beta chain amplification primers comprises a TCR beta chain complement subsequence that is substantially identical to a complement sequence of a constant region sequence of a TCR beta chain.

18. (canceled)

19. The method of claim 13, wherein the set of TCR beta chain amplification primers comprises a single TCR beta chain amplification primer.

20. The method of claim 19, wherein amplifying the beta TCR sequences comprises amplifying both beta chain 1 sequence and beta chain 2 sequence using the single TCR beta chain amplification primer.

21. The method of claim 20, wherein the single TCR beta chain amplification primer comprises SEQ ID NO: 6.

22. The method of claim 13, wherein each primer of the first set of TCR amplification primers comprises an anchor subsequence that is substantially identical to at least a subsequence of each TSO of the second RT primer set.

23. The method of claim 13, wherein a 3′ terminal subsequence of each primer of the first set of TCR amplification primers comprises a TG-3′ or a TGG-3′ sequence.

24. The method of claim 13, wherein the first set of TCR amplification primers comprises a single TCR amplification primer.

25. The method of claim 13, wherein amplifying the alpha and beta TCR sequences comprises using the three sets of TCR amplification primers
in a molar ratio of 2:1:1 of the first set of TCR amplification primers:the set of TCR alpha chain amplification primers:the set of TCR beta chain amplification primers; or
in a ratio of 2:1:1 of a weight of the first set of TCR amplification primers:a weight of the set of TCR alpha chain amplification primers:a weight of the set of TCR beta chain amplification primers.

26. (canceled)

27. The method of claim 13, wherein each primer of the first set of TCR amplification primers comprises SEQ ID NO: 96, each primer of the set of TCR alpha chain amplification primers comprises SEQ ID NO: 97 and each primer of the set of TCR beta chain amplification primers comprises SEQ ID NO: 98.

28. The method of claim 13, wherein amplifying the alpha and beta TCR sequences comprises amplifying the alpha TCR sequences, beta chain 1 sequence, and beta chain 2 sequence using the single TCR alpha chain amplification primer, the single beta chain amplification primer, and the single TCR amplification primer.

29. (canceled)

30. (canceled)

31. (canceled)

32. The method of claim 1, further comprising, before placing the T cell in the lysis solution, selecting the T cell based on
cell-surface expression of a marker selected from CD3, CD4, CD8, CD45RO, CD45RA, CCR7, CD62L, PD-1, CD137, or any combination thereof; or
expression by the T cell of one or more cytokines.

33. (canceled)

34. (canceled)

35. (canceled)

36. (canceled)

37. (canceled)

38. (canceled)

39. A kit comprising:
a first RT primer set comprising one or more first RT primers;
a second RT primer set comprising one or more second RT primers; and
a third RT primer set comprising one or more third RT primers;
a first set of TCR amplification primers;
a set of TCR alpha chain amplification primers; and
a set of TCR beta chain amplification primers.

40. (canceled)

41. (canceled)

42. (canceled)

43. A method for paired amplification of predetermined paired sequences of interest from a single cell, the method comprising:
placing a single cell into a cell lysis solution to provide a cell lysate comprising RNA;
generating first strand cDNA from the RNA;
amplifying the first strand cDNA to provide amplified cDNA; and
amplifying the predetermined paired sequences of interest from the amplified cDNA in a single reaction,
wherein the paired sequences are amplified using three sets of gene specific amplification primers.

44. (canceled)
